(19) United States
(12) Patent Application Publication
(10) Pub. No.: US 2025/0161254 A1
(43) Pub. Date: May 22, 2025

(54) USE OF RETINOIDS FOR TREATMENT OF ATRIAL FIBRILLATION

(71) Applicant: NEW YORK UNIVERSITY, New York, NY (US)

(72) Inventors: David S. PARK, New York, NY (US); Junhua XIAO, Parkton, MD (US); Naoko YAMAGUCHI, New York, NY (US)

(73) Assignee: NEW YORK UNIVERSITY, New York, NY (US)

(21) Appl. No.: 18/835,261

(22) PCT Filed: Feb. 3, 2023
(86) PCT No.: PCT/US23/12263

§ 371 Date: Aug. 1, 2024

Related U.S. Application Data
(60) Provisional application No. 63/306,955, filed on Feb. 4, 2022.

(51) Int. Cl. A61K 31/203; A61K 9/00; A61P 9/06; G01N 33/50

(52) U.S. Cl. CPC A61K 31/203; A61K 9/0019; A61P 9/06; G01N 33/50

(57) ABSTRACT

The invention relates to methods for treating atrial fibrillation or for preventing or reversing atrial remodeling comprising administering retinoids, such as, for example, all-trans retinoic acid (ATRA).

### CROSS-REFERENCE TO RELATED APPLICATIONS

This application claims priority to U.S. Provisional Patent Application No. 63/306,955, filed Feb. ^{4, 2022}, the disclosure of which is herein incorporated by reference in its entirety.

### FIELD OF THE INVENTION

The invention relates to methods for treating atrial fibrillation or for preventing or reversing atrial remodeling comprising administering retinoids, such as, for example, all-trans retinoic acid (ATRA).

### BACKGROUND

Atrial fibrillation (AF) is the most common arrhythmia encountered in clinical practice, with an estimated 2.7-6.1 million people affected in the US alone.2,3 AF negatively impacts all cardiovascular outcomes, increasing stroke risk, morbidity, mortality, and hospitalizations.^{4,5} The annual costs associated with AF hospitalizations in the US are estimated at $6.65 billion annually.7 The two main therapeutic options for rhythm control of AF are anti-arrhythmic drugs (AADs) and catheter ablation therapy. AADs have low efficacy at maintaining sinus rhythm and have lethal proarrhythmic side effects. Catheter ablation is more effective at maintaining sinus rhythm, but the procedure is invasive with the potential for major adverse events.9-11 Importantly, neither therapy addresses the underlying left atrial myopathy that gives rise to AF and promotes its progression to persistent form.

### SUMMARY OF THE INVENTION

In one aspect, the invention relates to a method for treating atrial fibrillation in a subject in need thereof, the method comprising administering to the subject a therapeutically effective amount of a retinoid or a pharmaceutically acceptable salt thereof. In certain embodiments, the subject has been determined to be deficient for said retinoid.

In another aspect, the invention relates to a method for treating atrial fibrillation in a subject in need thereof, comprising: a) determining the level of a retinoid in a serum sample obtained from the subject; b) comparing the level of the retinoid determined in step (a) to a control level of said retinoid; c) administering to the subject determined to be deficient for said retinoid based on the comparison in step (b) a therapeutically effective amount of said retinoid or a pharmaceutically acceptable salt thereof. In certain embodiments, the therapy with said retinoid or pharmaceutically acceptable salt thereof is continued until the subject is no longer determined to be deficient for said retinoid by repeating steps (a)-(b).

In certain embodiments of any of the above methods, the subject has been diagnosed with heart failure. In certain embodiments of any of the above methods, the subject has been diagnosed with hypertension.

In another aspect, the invention relates to a method for preventing or reversing atrial remodeling in a subject in need thereof, the method comprising administering to the subject a therapeutically effective amount of a retinoid or a pharmaceutically acceptable salt thereof. In certain embodiments, the subject has been determined to be deficient for said retinoid.

In another aspect, the invention relates to a method for preventing or reversing atrial remodeling in a subject in need thereof, comprising: a) determining the level of a retinoid in a serum sample obtained from the subject; b) comparing the level of the retinoid determined in step (a) to a control level of said retinoid; c) administering to the subject determined to be deficient for said retinoid based on the comparison in step (b) a therapeutically effective amount of said retinoid or a pharmaceutically acceptable salt thereof. In certain embodiments, the therapy with said retinoid or pharmaceutically acceptable salt thereof is continued until the subject is no longer determined to be deficient for said retinoid by repeating steps (a)-(b).

In certain embodiments of the above two methods, the subject has been diagnosed with acute onset heart failure.

In certain embodiments of any of the above methods, the retinoid is a retinoic acid. In certain embodiments of any of the above methods, the retinoid is all-trans retinoic acid (ATRA).

In certain embodiments of any of the above methods, the subject is human. In certain embodiments of any of the above methods, the subject is more than 65 years of age.

In certain embodiments of any of the above methods, the retinoid or pharmaceutically acceptable salt thereof is administered or delivered to the atrium.

In certain embodiments of any of the above methods, the retinoid or pharmaceutically acceptable salt thereof is administered in combination with one or more additional therapeutic agents.

These and other aspects of the present invention will be apparent to those of ordinary skill in the art in the following description, claims and drawings.

### BRIEF DESCRIPTION OF THE DRAWINGS

FIGS. 1A-1C depict that RNA-seq analysis shows differentially expressed genes in the mouse left atrium (LA) with transverse aortic constriction (TAC) banding and Angiotensin II (AngII) infusion.1 (FIG. 1A) Venn diagram displaying overlap of 3364 differentially expressed genes between TAC and AngII experiments. (FIG. 1B) Pairwise correlation between log fold change of differential gene expression in AngII vs. TAC banded experiments (R=0.88, P<2.2×10-16). (FIG. 1C) KEGG pathway analysis of the upregulated genes involved in signaling pathways from the group of 3364 overlapping genes in FIG. 1A.

FIG. 2 shows a retinoic acid (RA) synthesis pathway. Circulating retinol is converted to all-trans-RA (ATRA) via successive oxidative steps. ALDH1A2 (also referred to as RALDH2) catalyzes the final step of RA biosynthesis. RA then activates nuclear retinoic acid receptor (RAR) and retinoid X receptor (RXR) that bind to retinoic acid response elements (RAREs) as heterodimers to activate target gene transcription. ATRA levels are tightly regulated by CYP26A1, CYP26B1, and CYP26C1, which catabolize ATRA for elimination.

FIGS. 3A-3B show RNA expression profiling of human left atrial tissue. (FIG. 3A) Lower ALDHIA2 expression in the left atrium (LA) correlates with reduced left ventricular ejection fraction (LVEF), indicating reduced ATRA biosynthesis in the LA with heart failure. (FIG. 3B) Higher CYP26B1 expression in the LA associates with persistent atrial fibrillation (AF), indicating that increased ATRA degradation contributes to LA remodeling that gives rise to AF.

FIGS. 4A-4E show electrocardiograment of trans-aortic constriction (TAC) banded mice treated with vehicle (V) or ATRA 10 mg/kg administered daily via intraperitoneal injection for a two-week study period. (FIG. 4A) Heart rate (HR) is increased in TAC+V and TAC+ATRA cohorts. (FIG. 4B) PR interval is shortened in TAC+V and TAC+ATRA cohorts. ATRA does not significantly alter PR interval in the TAC+ATRA vs TAC+V cohort. (FIG. 4C) P wave duration is prolonged in the TAC+V cohort. ATRA treatment prevents P wave prolongation in the TAC+ATRA cohort. QRS duration (FIG. 4D) and HR corrected QT (QTc) interval (FIG. 4E) are prolonged equally in TAC+V and TAC+ATRA cohorts. n=15-18 per group. Data represent mean+S.E.M. One-way ANOVA was used followed by Tukey's test for multiple comparisons to compare differences between experimental groups. * p-value<0.05, ** p-value<0.005, *** p-value<0.001, **** p-value<0.0001

FIGS. 5A-5E show cardiac structural and functional assessment using transthoracic echocardiogram in Sham surgery versus trans-aortic constriction (TAC) banded cohorts treated with Vehicle (V) control versus ATRA 10 mg/kg administered daily via intraperitoneal injection over a two-week time period. (FIG. 5A) M-mode comparison of Sham+V, Sham+ATRA, TAC+V, TAC+ATRA of the left ventricle (LV). LV wall thickness represented by arrows is increased in both TAC+V and TAC+ATRA cohorts. (FIG. 5B) Normalized LV wall thickness. (FIG. 5C) LV fractional shortening (FS). FS is equally reduced in the TAC+V and TAC+ATRA hearts. (FIG. 5D) LV strain measured using speckle tracking algorithm. LV strain is equally reduced in TAC+V and TAC+ATRA hearts. (FIG. 5E) Reservoir strain values were measured in the left atrium (LA). 12 TAC+V LA showed reduced reservoir strain. TAC+ATRA hearts showed significant improvement in reservoir strain parameters. All measurements made in parasternal long axis view. All scale bars are 1 mm. n=6 in Sham+V and Sham+ATRA groups, n=13 in TAC+V and TAC+ATRA groups for (FIG. 5B-5D). n=13 in each group for (FIG. 5E). One-way ANOVA was used followed by Tukey's test for multiple comparisons to compare differences between experimental groups. * p-value<0.05, ** p-value<0.005, *** p-value<0.001, **** p-value<0.0001

FIGS. 6A-6E show cardiac optical mapping of TAC banded mice treated with ATRA. Optical mapping of Langendorff-perfused hearts from Sham+V, TAC+V, and TAC+ATRA hearts. (FIG. 6A) Representative left atrial activation maps at 37° C. Hearts were paced at 100 ms basic cycle length (BCL) from the right atria. (FIG. 6B) Calculated LA conduction velocity (CV). (FIG. 6C) Calculated left ventricular CV (FIG. 6D) ECG tracing of sustained atrial arrythmia in TAC+V heart after atrial burst pacing protocol.⁶ (FIG. 6E) Left atrial activation map of sustained atrial arrythmia shown in panel (FIG. 6D). Isochrones are drawn 1 ms apart. Data represent mean+S.E.M. n=4-5 per group. One-way ANOVA was used followed by Tukey's test for multiple comparisons to compare differences between experimental groups. * p-value<0.05, ** p-value<0.005, *** p-value<0.001, **** p-value<0.0001

FIG. 7 shows histology of TAC banded hearts treated with ATRA 10 mg/kg daily via intraperitoneal injection over a two-week time period. (A) Trichrome staining of left atrial sections. (B) Higher magnification views of selected atrial regions from above panel in (A). Treatment with ATRA limits interstitial and perivascular fibrosis associated with TAC banding. (C) Measured collagen volume fraction by ImageJ analysis. n=5 per group. Data represent mean+S.E.M. One-way ANOVA was used followed by Tukey's test for multiple comparisons to compare differences between experimental groups. * p-value<0.05, ** p-value<0.005, *** p-value<0.001, **** p-value<0.0001

FIG. 8 shows Connexin-43 (Cx43) expression in TAC banded hearts treated with ATRA. Sham+V, Sham+ATRA, TAC+V, TAC+ATRA hearts were perfusion-fixed and paraffin-embedded. Sections were probed with antibodies to Cx43, N-cadherin (N-CAD) and DAPI nuclear stain. Sham+V and Sham+ATRA hearts show similar expression and localization of Cx43 at the intercalated discs, as evidence by colocalization of Cx43 and N-CAD. TAC+V LA show diminished Cx43 expression and relocalization of Cx43 to the lateral membranes. TAC+ATRA shows robust Cx43 expression at the intercalated discs. Scale bars: 20 um

FIGS. 9A-9E show ATRA effect on gene expression profile in TAC banded hearts. (FIG. 9A) Heatmap of differentially expressed genes (DEG, p<0.05) between TAC+ATRA and TAC+V (n=5 per group). (FIG. 9B) Venn diagram of total number of upregulated or downregulated DEG (p<0.05 in each group) in [TAC+ATRA_vs_TAC+V] vs [TAC_vs_Sham] by Venny2.1. Reciprocally expressed genes in [TAC+ATRA_vs_TAC+V] vs [TAC_vs_Sham] (952 upregulated and 1035 downregulated genes) were used for functional analysis. (FIG. 9C, FIG. 9D) Reactome pathway and Gene Ontology (GO) classifications in Biological Process category of the upregulated (FIG. 9C) and downregulated (FIG. 9D) DEG analyzed by Enrichr. Dash line represents -log 10 (P=0.05). (#) number of annotated genes. (FIG. 9E) Heatmaps of genes involved in the curated lists of I-VI from functional analysis showing reciprocal changes between TAC vs Sham and TAC+ATRA vs TAC

FIGS. 10A-10H show electrocardiogramd histological assessment of trans-aortic constriction (TAC) banded mice with delayed vehicle (dV) versus delayed ATRA (dATRA) treatment. In this experiment, study mice were TAC banded and left untreated for 2 weeks. At 2 weeks, TAC mice were treated with dV or dATRA for two weeks, making the total study period 4 weeks. (FIG. 10A) Heart rate (HR), (FIG. 10B) PR interval, (FIG. 10D) QRS duration, and (FIG. 10E) QTc interval are unchanged by dATRA treatment. (FIG. 10C) dATRA treatment significantly reduces the P wave duration. (FIG. 10F) Trichrome staining of paraffin-embedded left atrial sections. (FIG. 10G) Higher magnification views of selected atrial regions from above panel in FIG. 10F. Delayed treatment with ATRA reverses interstitial and perivascular fibrosis associated with TAC banding. (FIG. 10H) Measured collagen volume fraction by ImageJ analysis. n=5-8 per group, even distribution of genders. Data represent mean+S.E.M. One-way ANOVA was used followed by Tukey's test for multiple comparisons to compare differences between experimental groups. * p-value<0.05, ** p-value<0.005, *** p-value<0.001, **** p-value<0.0001

FIGS. 11A-11D show electrocardiograment of Sham surgery group versus trans-aortic constriction (TAC) banded mice treated with vehicle (V) or ATRA at various doses (2 mg/kg, 5 mg/kg, 10 mg/kg, and 20 mg/kg administered daily via intraperitoneal injection over a two-week time period). Although heart rate (HR) (FIG. 11A) and PR interval (FIG. 11B) and QRS duration (FIG. 11D) is not significantly changed in TAC+V versus TAC+ATRA cohorts over an ATRA dose range of 2 mg/kg-10 mg/kg, shortening of P wave duration (FIG. 11C) is ATRA dose dependent. Maximal P wave duration shortening is seen at an ATRA dose of 10m/kg, with no further shortening with the 20 mg/kg dose. Notably, ATRA 20 mg/kg demonstrated increased ventricular ectopy, indicating potential cardiotoxicity at this high dose. n=8-10 per group. Data represent mean #S.E.M. One-way ANOVA was used followed by Tukey's test for multiple comparisons to compare differences between experimental groups. * p-value<0.05, ** p-value<0.005, *** p-value<0.001, **** p-value<0.0001

### DETAILED DESCRIPTION

The present invention relates to methods for treating atrial fibrillation or for preventing or reversing atrial remodeling comprising administering retinoids, such as, for example, all-trans retinoic acid (ATRA).

### Definitions

To facilitate an understanding of the principles and features of the various embodiments of the invention, various illustrative embodiments are explained below. Although exemplary embodiments of the invention are explained in detail, it is to be understood that other embodiments are contemplated. Accordingly, it is not intended that the invention is limited in its scope to the details of construction and arrangement of components set forth in the following description or examples. The invention is capable of other embodiments and of being practiced or carried out in various ways. Also, in describing the exemplary embodiments, specific terminology will be resorted to for the sake of clarity.

The term “about” or “approximately” means within a statistically meaningful range of a value. Such a range can be within an order of magnitude, preferably within 50%, more preferably within 20%, still more preferably within 10%, and even more preferably within 5% of a given value or range. The allowable variation encompassed by the term “about” or “approximately” depends on the particular system under study, and can be readily appreciated by one of ordinary skill in the art.

The terms “a,” “an,” and “the” do not denote a limitation of quantity, but rather denote the presence of “at least one” of the referenced item.

The terms “patient”, “individual”, “subject”, and “animal” are used interchangeably herein and refer to mammals, including, without limitation, human and veterinary animals (e.g., cats, dogs, cows, horses, sheep, pigs, etc.) and experimental animal models. In a preferred embodiment, the subject is a human.

The terms “treat” or “treatment” of a state, disorder or condition include: (1) preventing or delaying the appearance of at least one clinical or sub-clinical symptom of the state, disorder or condition developing in a subject that may be afflicted with or predisposed to the state, disorder or condition but does not yet experience or display clinical or subclinical symptoms of the state, disorder or condition; or (2) inhibiting the state, disorder or condition, i.e., arresting, reducing or delaying the development of the disease or a relapse thereof (in case of maintenance treatment) or at least one clinical or sub-clinical symptom thereof; or (3) relieving the disease, i.e., causing regression of the state, disorder or condition or at least one of its clinical or sub-clinical symptoms. The benefit to a subject to be treated is either statistically significant or at least perceptible to the patient or to the physician.

An “effective amount” applied to dose or amount refers to that quantity of a compound or pharmaceutical composition that is sufficient to result in a desired activity upon administration to a subject in need thereof. Note that when a combination of active ingredients is administered, the effective amount of the combination may or may not include amounts of each ingredient that would have been effective if administered individually. The exact amount required will vary from subject to subject, depending on the species, age, and general condition of the subject, the severity of the condition being treated, the particular drug or drugs employed, the mode of administration, and the like.

A “therapeutically effective amount” of a compound described herein is an amount sufficient to provide a therapeutic benefit in the treatment of a state, disorder or condition or to delay or minimize one or more symptoms associated with the state, disorder or condition. A therapeutically effective amount of a compound means an amount of therapeutic agent, alone or in combination with other therapies, which provides a therapeutic benefit in the treatment of the condition. The term “therapeutically effective amount” can encompass an amount that improves overall therapy, reduces or avoids symptoms or causes of the condition, and/or enhances the therapeutic efficacy of another therapeutic agent.

In certain embodiments, the pharmaceutically acceptable salts include salts of acidic or basic groups present in compounds of the present disclosure. As used herein, the term “pharmaceutically acceptable salt” means those salts of compounds of the invention that are safe for application in a subject. Pharmaceutically acceptable acid salts include, but are not limited to, hydrochloride, hydrobromide, hydroiodide, nitrate, sulfate, bisulfate, phosphate, acid phosphate, isonicotinate, acetate, lactate, salicylate, citrate, tartrate, pantothenate, bitartrate, ascorbate, succinate, maleate, gentisinate, fumarate, gluconate, glucaronate, saccharate, formate, benzoate, glutamate, methanesulfonate, ethanesulfonate, benzensulfonate, p-toluenesulfonate and pamoate (i.e., 1,11-methylene-bis-(2-hydroxy-3-naphthoate)) salts. Certain compounds of the present disclosure can form pharmaceutically acceptable salts with various amino acids. Suitable base salts include, but are not limited to, aluminum, calcium, lithium, magnesium, potassium, sodium, zinc, and diethanolamine salts. Lists of suitable salts are found in Remington's Pharmaceutical Sciences, 17th ed., Mack Publishing Company, Easton, Pa., 1985, p. 1418 and Berge, S M et al, Journal of Pharmaceutical Science, 1977, 66, 1, 1-19.

In the context of the field of medicine, the term “prevent” encompasses any activity which reduces the burden of mortality or morbidity from disease. Prevention can occur at primary, secondary and tertiary prevention levels. While primary prevention avoids the development of a disease, secondary and tertiary levels of prevention encompass activities aimed at preventing the progression of a disease and the emergence of symptoms as well as reducing the negative impact of an already established disease by restoring function and reducing disease-related complications.

The methods and techniques of the present invention are generally performed according to conventional methods well known in the art and as described in various general and more specific references that are cited and discussed throughout the present specification unless otherwise indicated. See, e.g., Sambrook et al., Molecular Cloning: A Laboratory Manual, 2d ed., Cold Spring Harbor Laboratory Press, Cold Spring Harbor, N.Y. (1989) and Ausubel et al., Current Protocols in Molecular Biology, Greene Publishing Associates (1992), and Harlow and Lane Antibodies: A Laboratory Manual, Cold Spring Harbor Laboratory Press, Cold Spring Harbor, N.Y. (1990), which are incorporated herein by reference. Enzymatic reactions and purification techniques are performed according to manufacturer's specifications, as commonly accomplished in the art or as described herein. The nomenclatures used in connection with, and the laboratory procedures and techniques of, analytical chemistry, synthetic organic chemistry, and medicinal and pharmaceutical chemistry described herein are those well-known and commonly used in the art. Standard techniques are used for chemical syntheses, chemical analyses, pharmaceutical preparation, formulation, and delivery, and treatment of patients.

Also, in describing the exemplary embodiments, terminology will be resorted to for the sake of clarity. It is intended that each term contemplates its broadest meaning as understood by those skilled in the art and includes all technical equivalents which operate in a similar manner to accomplish a similar purpose.

### Methods of the Invention

In one aspect, the invention relates to a method for treating atrial fibrillation in a subject in need thereof, the method comprising administering to the subject a therapeutically effective amount of a retinoid or a pharmaceutically acceptable salt thereof. In certain embodiments, the subject has been determined to be deficient for said retinoid.

In another aspect, the invention relates to a method for treating atrial fibrillation in a subject in need thereof, comprising: a) determining the level of a retinoid in a serum sample obtained from the subject; b) comparing the level of the retinoid determined in step (a) to a control level of said retinoid; c) administering to the subject determined to be deficient for said retinoid based on the comparison in step (b) a therapeutically effective amount of said retinoid or a pharmaceutically acceptable salt thereof. In certain embodiments, the therapy with said retinoid or pharmaceutically acceptable salt thereof is continued until the subject is no longer determined to be deficient for said retinoid by repeating steps (a)-(b).

In another aspect, the invention relates to a method for preventing or reversing atrial remodeling in a subject in need thereof, the method comprising administering to the subject a therapeutically effective amount of a retinoid or a pharmaceutically acceptable salt thereof. In certain embodiments, the subject has been determined to be deficient for said retinoid.

In another aspect, the invention relates to a method for preventing or reversing atrial remodeling in a subject in need thereof, comprising: a) determining the level of a retinoid in a serum sample obtained from the subject; b) comparing the level of the retinoid determined in step (a) to a control level of said retinoid; c) administering to the subject determined to be deficient for said retinoid based on the comparison in step (b) a therapeutically effective amount of said retinoid or a pharmaceutically acceptable salt thereof. In certain embodiments, the therapy with said retinoid or pharmaceutically acceptable salt thereof is continued until the subject is no longer determined to be deficient for said retinoid by repeating steps (a)-(b).

Quantification of retinoids can be done with any methods known in the art and as described in various general and more specific references that are cited and discussed throughout the present specification unless otherwise indicated. See, e.g., Yang, N., et al. Cardiac retinoic acid levels decline in heart failure. JCI Insight 6 (2021). In certain embodiments, the retinoids can be quantified, for example, by using LC-MRM (liquid chromatography with multiple reaction monitoring), UHPLC-MS/MS (ultra-high-performance liquid chromatography), HPLC/MS″, LC-MS/MS, GC/MS, or LC/diode array detector-atmospheric pressure chemical ionization/MS/MS.

In certain non-limiting embodiments, the retinoid is retinol (Vitamin A), retinal (retinaldehyde), retiferol, tretinoin (retinoic acid), isotretinoin, alitretinoin (9-cis-retinoic acid), etretinate, acitretin, adapalene, bexarotene, tazarotene, trifarotene. In certain embodiments, the retinol is a retinol isomer. In certain embodiments, the retinol isomer is all-trans-retinol, 13-cis-retinol, 11-cis-retinol, 9-cis-retinol, 3,4-didehydro-retinol, 3,4-didehydro-13-cis-retinol; 3,4-didehydro-11-cis-retinol; or 3,4-didehydro-9-cis-retinol.

In certain non-limiting embodiments, the retinoid is a compound comprising a retinoid structure, a retinoid metabolite, or an agent that can be metabolized into a retinoid or retinoid metabolite. A retinoid further includes a compound that is an analog or mimic of a retinoid or a retinoid metabolite, or an agent that can be metabolized into an analog or mimic of a retinoid or a retinoid metabolite. In certain embodiments the retinoid can be any retinoid disclosed in U.S. Pat. Nos. 5,648,563; 5,648,385; 5,618,839; 5,559,248; 5,616,712; 5,616,597; 5,602,135; 5,599,819; 5,556,996; 5,534,516; 5,516,904; 5,498,755; 5,470,999; 5,468,879; 5,455,265; 5,451,605; 5,426,118; 5,407,937; 5,399,586; 5,399,561; 5,391,753.

In certain embodiments, the retinoid is a retinoic acid. In certain embodiments, the retinoid is all-trans retinoic acid (ATRA).

In certain embodiments, ATRA is

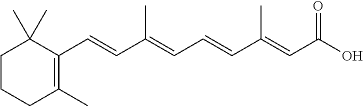 [SMILES: CC1=C(/C=C/C(C)=C/C=C/C(C)=C/C(=O)O)C(C)(C)CCC1]

or a pharmaceutically acceptable salt thereof.

In certain embodiments, the subject has been diagnosed with heart failure. In certain embodiments, the subject has been diagnosed with hypertension. In certain embodiments, the subject is diagnosed with acute onset heart failure.

In certain embodiments, the subject is human. In certain embodiments, the subject is more than 65 years of age.

In certain embodiments, the retinoid or pharmaceutically acceptable salt thereof is administered or delivered to the atrium.

In certain embodiments, the retinoid or pharmaceutically acceptable salt thereof is administered in combination with one or more additional therapeutic agents.

The administration route may be any mode of administration known in the art, including but not limited to injection into involved tissue, intraarterially, intravenously, via an implanted device, parenterally, topically, subcutaneously, intradermally, transdermally (e.g., by transdermal patch), via intracorporal application during surgery, intramuscularly, intraperitoneally, buccally, intrathecally, intracranially, or orally. In a preferred embodiment, a retinoid or pharmaceutically acceptable salt thereof is administered or delivered to the atrium.

A retinoid or pharmaceutically acceptable salt thereof of the present invention may be encapsulated or otherwise protected against gastric or other secretions, if desired.

In certain embodiments, a retinoid or pharmaceutically acceptable salt thereof of the present invention may be administered in conjunction with other treatments. In certain embodiments, a retinoid or pharmaceutically acceptable salt thereof of the present invention may be administered in conjunction with treatment for, but not limited to, atrial fibrillation, heart failure, hypertension and/or acute onset heart failure. In certain embodiments the other treatment can be, but is not limited to, cardioversion, including electrical and/or drug cardioversion, surgery, coronary bypass surgery, heart valve repair or replacement, catheter procedures, use of implantable cardioverter-defibrillators (ICDs), cardiac resynchronization therapy (CRT), use of ventricular assist devices (VADs), heart transplantation, palliative care, beta blockers (e.g., atenolol, metoprolol or bisoprolol), calcium channel blockers (e.g., amlodipine or diltiazem), digoxin, anti-arrhythmic medications, blood thinners (e.g., warfarin, apixaban, dabigatran, edoxaban or rivaroxaban), angiotensin-converting enzyme (ACE) inhibitors (e.g., enalapril, benazepril, lisinopril or captopril), angiotensin II receptor blockers (e.g., losartan, valsartan or candesartan), diuretics (e.g., furosemide), aldosterone antagonists (e.g., spironolactone or eplerenone), positive inotropes, hydralazine and isosorbide dinitrate (BiDil), vericiguat, water pills (diuretics), alpha blockers (e.g., doxazosin or prazosin), alpha-beta blockers (e.g., carvedilol or labetalol), renin inhibitors (e.g., aliskiren), vasodilators (e.g., hydralazine or minoxidil), central-acting agents (e.g., clonidine, guanfacine or methyldopa) and combinations thereof.

The dosage administered will be dependent upon the route of administration, age, health, and weight of the recipient, kind of concurrent treatment, if any, frequency of treatment, and the nature of the effect desired.

Effective doses of retinoids or pharmaceutically acceptable salts thereof for therapeutic uses discussed above may be determined using methods known to one skilled in the art. Effective doses may be determined, preferably in vitro, in order to identify the optimal dose range using any of the various methods described herein. In one embodiment, an aqueous solution of a retinoid or pharmaceutically acceptable salt thereof is administered by intraperitoneal injection. Each dose may range from about 0.001 μg/kg body weight to about 100 mg/kg body weight, or more preferably, from about 0.1 μg/kg to 20 mg/kg body weight. The dosing schedule may vary from one time only to once a week to daily or twice (or more) daily depending on a number of clinical factors.

A suitable, non-limiting example of a dosage of a retinoid or pharmaceutically acceptable salt thereof according to the present invention or a composition comprising such a retinoid or pharmaceutically acceptable salt thereof, is from about 1 ng/kg to about 1000 mg/kg, such as from about 1 mg/kg to about 100 mg/kg, including from about 5 mg/kg to about 50 mg/kg. Other representative dosages of a compound or a composition of the present invention include about 1 mg/kg, 5 mg/kg, 10 mg/kg, 15 mg/kg, 20 mg/kg, 25 mg/kg, 30 mg/kg, 35 mg/kg, 40 mg/kg, 45 mg/kg, 50 mg/kg, 60 mg/kg, 70 mg/kg, 80 mg/kg, 90 mg/kg, 100 mg/kg, 125 mg/kg, 150 mg/kg, 175 mg/kg, 200 mg/kg, 250 mg/kg, 300 mg/kg, 400 mg/kg, 500 mg/kg, 600 mg/kg, 700 mg/kg, 800 mg/kg, 900 mg/kg, or 1000 mg/kg.

In certain embodiment, the retinoid or pharmaceutically acceptable salt thereof, is administered at a dose of about 2 mg/kg, 5 mg/kg, 10 mg/kg, or 20 mg/kg. In certain embodiment, the retinoid or pharmaceutically acceptable salt thereof, is administered intraperitoneally.

In certain embodiments, the retinoid or pharmaceutically acceptable salt thereof may be administered hourly, daily, weekly, monthly, yearly or as a onetime delivery. In certain embodiments, the retinoid or pharmaceutically acceptable salt thereof may be administered daily via intraperitoneal injection over a one-week, two-week, three-week or four-week time period.

In certain embodiments of the present invention, the methods may further comprise administering a pharmaceutically acceptable carrier to the subject during the administration of the retinoid or pharmaceutically acceptable salt thereof. The carrier may be a diluent, an aerosol, a topical carrier, an aqueous solution, a nonaqueous solution or a solid carrier. As used herein, the term “suitable pharmaceutically acceptable carrier” encompasses any of the standard pharmaceutically accepted carriers, such as phosphate buffered saline solution, water, emulsions such as an oil/water emulsion or a triglyceride emulsion, various types of wetting agents, tablets, coated tablets and capsules.

### Examples

The present invention is also described and demonstrated by way of the following examples. However, the use of these and other examples anywhere in the specification is illustrative only and in no way limits the scope and meaning of the invention or of any exemplified term. Likewise, the invention is not limited to any particular preferred embodiments described here. Indeed, many modifications and variations of the invention may be apparent to those skilled in the art upon reading this specification, and such variations can be made without departing from the invention in spirit or in scope. The invention is therefore to be limited only by the terms of the appended claims along with the full scope of equivalents to which those claims are entitled.

### Example 1. Identification of ATRA as a Master Regulatory Switch that can Protect Against Pathological Atrial Remodeling

Atrial fibrillation (AF) is a multi-factorial disease, whereby genetic predisposition coupled with pathological stressors create the electrical and structural changes in the left atrium (LA) that promote AF. The maladaptive changes, which include conduction slowing, inflammation, and fibrosis, are known as atrial remodeling.12,13 The LA is particularly vulnerable to the remodeling process when exposed to pressure overload conditions that include hypertensive heart disease, valvular heart disease, and diastolic and systolic heart failure (HF). The remodeled atrium exhibits reduced conduction velocity (CV) as a result of decreased cardiac sodium channel Nav1.5 (encoded by Scn5a) expression resulting in decreased sodium current (INa),14 diminished expression and mis-localization of high conductance gap junction proteins Connexin 43 and 40 (Cx43 and Cx40, encoded by Gja1 and Gja5, respectively) from intercalated discs, 15,16 and increased fibrosis,17 which impairs conduction by disrupting muscle fiber continuity. INa is the principal determinant of membrane excitability in cardiomyocytes, and Cx43 and Cx40 gap junctions facilitate passive conductance between myocytes.18,19 The remodeling process tends to be progressive, resulting in an atrial myopathy that initiates AF and drives its progression from a paroxysmal (self-terminating) to persistent form.

As mentioned in the Background section, neither anti-arrhythmic drugs (AADs) nor catheter ablation therapy addresses the underlying left atrial myopathy that gives rise to atrial fibrillation (AF) and promotes its progression to persistent form. To address this critical unmet need, the present inventors used atrial developmental signaling pathways to identify a novel therapy that can prevent and reverse the pathological atrial remodeling processes causing AF.

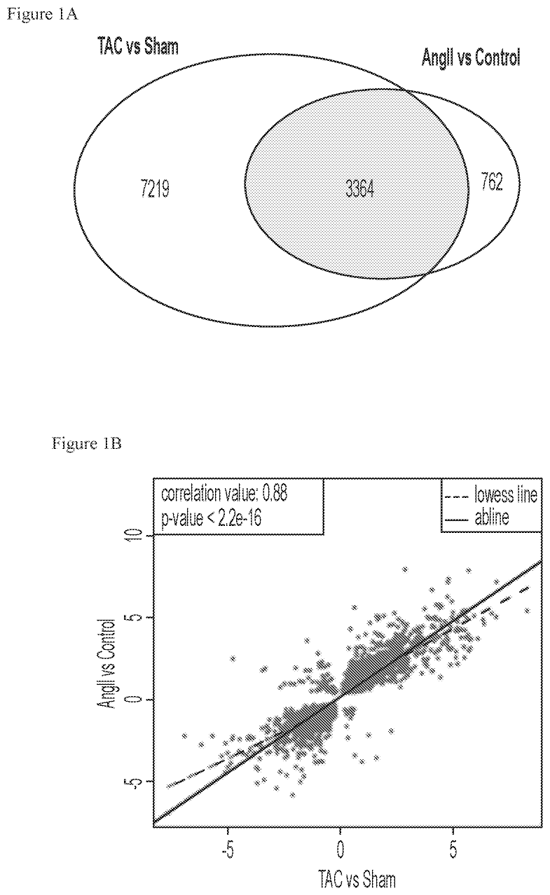

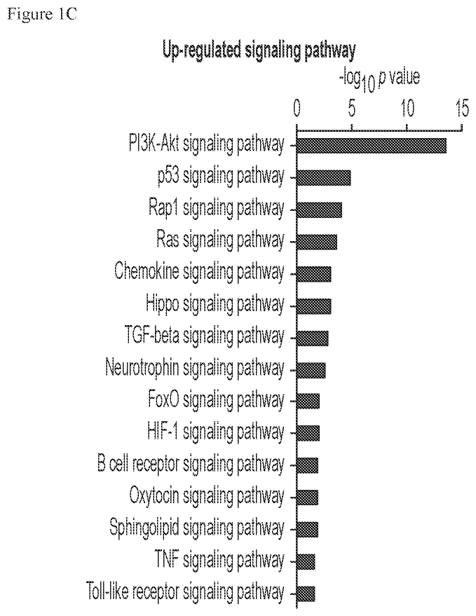

To better define the atrial remodeling process, the inventors performed comparative transcriptomic analysis of two cardiac pressure overload models, transaortic constriction (TAC) banding and angiotensin II (AngII) infusion (FIG. 1A). It was found that the LA undergoes remodeling in a highly reproducible manner (FIG. 1B). The major biological processes that were upregulated in both models involved pro-inflammatory pathways (chemokine signaling, transforming growth factor-beta (TGF-B) signaling, B cell receptor signaling, tumor necrosis factor (TNF) signaling, and Toll-like receptor (TLR) signaling) and profibrotic pathways (extracellular matrix (ECM) receptor interaction and focal adhesion). These pro-inflammatory and pro-fibrotic pathways underlie the molecular basis for structural remodeling. The major downregulated biological processes were metabolic pathways (respiratory transport chain in the mitochondria and fatty acid oxidation) and pathways that affect rapid conduction gene expression (adrenergic signaling in cardiomyocytes, arrhythmogenic cardiomyopathy, and ERBB signaling pathway). Downregulation of rapid conduction genes, such as Scn5a, Gja1/Cx43, and Gja5/Cx40, contributes to electrical remodeling. The highly uniform manner in which the LA remodels indicates that master regulatory switches are in place to prevent inflammation/fibrosis, metabolic derangement, and conduction disease. It was hypothesized herein that cardiac pressure overload turns off these regulatory switches, allowing pathological remodeling to ensue.

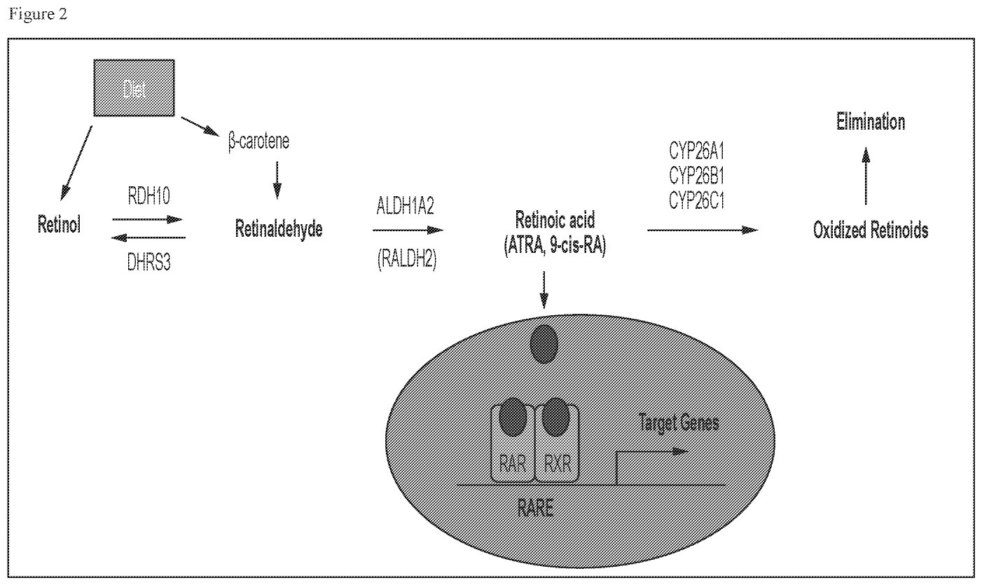

To identify these master regulatory switches that can serve as therapeutic targets for atrial remodeling, signaling pathways that are essential for atrial development were examined. Atrial myocytes derive from posterior second heart field (SHF) progenitor cells that contribute to the inflow tract of the linear heart tube. Retinoic acid signaling is critical for SHF progenitors to differentiate into atrial myocytes.21 Endogenous retinoids are derived from vitamin A through successive oxidative steps, generating the active metabolite all-trans retinoic acid (ATRA), the predominant form of retinoic acid (FIG. 2). Mice deficient in aldehyde dehydrogenase 1 family member A2 (ALDH1A2, also known as RALDH2), which oxidizes the final step in retinoic acid biosynthesis, die at embryonic day 10.5 (E10.5) and exhibit severe impairment in atrial outgrowth.20 Maternal supplementation with oral ATRA was able to rescue the atrial defects, indicating that the atria are responsive to circulating ATRA.20 On the other hand, chick embryos exposed to excess ATRA developed enlarged atria.22 The importance of retinoic acid in human atrial myocyte differentiation has been demonstrated using human embryonic stem cell-derived cardiomyocytes (hESC-CMs) and human-induced pluripotent stem cell-derived cardiomyocytes (hiPSC-CMs).23,24 Addition of retinoic acid effectively increased the population of atrial myocytes from a progenitor pool. 23-25

Based on the importance of ATRA signaling in atrial myocyte differentiation and chamber formation, it was hypothesized herein that ATRA would have an essential role in maintenance of atrial electrical and structural homeostasis and in cardio-protection against pathological atrial remodeling. In support of the maintenance hypothesis, Aldh1a1 and Aldh1a2 (ATRA biosynthesis) expression levels were 8-fold and 10-fold higher, respectively, in atrial myocytes compared to ventricular myocytes in the postnatal day 21 (P21) heart. Whereas Cyp26b1 (ATRA degradation) expression levels were 14-fold lower in atrial myocytes compared to ventricular myocytes in the P21 heart. These data suggest that continued ATRA enrichment is necessary to maintain the postnatal atrium. In support of the role of ATRA in cardio-protection, ATRA levels decline by 39% and 32% in the ventricular myocardium of patients with HF and in guinea pig HF models, respectively.26 ATRA treatment was able to improve left ventricular systolic function and reduce ventricular fibrosis in the guinea pig HF model. Consistent with this finding, ATRA reduces proliferation, collagen secretion, and TGF-β production in dissociated neonatal rat cardiac fibroblasts exposed to AngII.27 In the diabetic kidney, ATRA blocks production of inflammatory cytokines, chemokines, adhesion molecules, and growth factors by downregulating TLR signaling pathways that involve the transcription factor, NF-KB.28 Although ATRA presents itself as a regulatory switch that can protect against pathological atrial remodeling, there are no studies that have explored the role of ATRA signaling in the postnatal atrium.

The present inventors have uncovered an unrecognized role of ATRA in maintaining the electrical and structural integrity of the LA under pressure overload conditions. Analysis of the Cleveland Clinic Biobank reveals that ALDHIA2, the essential ATRA biosynthetic enzyme, is downregulated in the LA of patients with reduced left ventricular ejection fraction (LVEF), and that CYP26B1, which catalyzes ATRA degradation, is upregulated in the LA of patients with persistent AF. These data suggest that ATRA downregulation contributes to the atrial remodeling process that gives rise to AF. In support of this hypothesis, it was shown herein that ATRA treatment is highly effective in preventing electrical and structural remodeling in the LA due to cardiac pressure overload. Using TAC banding as a model for cardiac pressure overload, it was shown herein that ATRA treatment normalizes atrial conduction properties, improves left atrial functional parameters, and prevents atrial fibrosis. In addition, ATRA therapy prevented Cx43 gap junction remodeling in the LA of TAC banded hearts, as evidenced by normalization of Cx43 expression at intercalated discs. RNA-seq analysis revealed that ATRA treatment upregulates genes important for atrial conduction and metabolism and downregulates pro-inflammatory signaling pathways (AngII signaling, TLR signaling, platelet-derived growth factor (PDGF) signaling, TNF signaling, and interleukin 1b (IL-1b) signaling) and pathways involved in ECM deposition. Lastly, it was shown herein that ATRA treatment can reverse left atrial fibrosis and improve atrial conduction parameters even when initiated two weeks after TAC banding, which has major clinical implications for patients presenting with HF.

Having implicated ATRA as a master regulatory switch that prevents and reverses atrial remodeling during cardiac pressure overload, the underlying mechanisms of ATRA-mediated protection are investigated. The present hypotheses are that i) endogenous ATRA maintains electrical and structural integrity of the adult left atrium, ii) cardiac pressure overload diminishes endogenous ATRA levels in the LA to promote pathological remodeling, iii) therapeutic ATRA blocks key pro-inflammatory cytokine/chemokine/growth factor production pathways that drive fibrosis, and iv) therapeutic ATRA normalizes left atrial conduction in part by restoring sodium and gap junctional conductances. To validate these hypotheses, the following studies are performed:

1: Studies of the role of endogenous ATRA in maintaining normal atrial physiology, the impact of pressure overload on endogenous ATRA levels, and the therapeutic potential of raising endogenous ATRA levels to prevent TAC-induced LA remodeling.

2: Studies to define the anti-inflammatory mechanisms by which ATRA prevents and reverses atrial fibrosis and studies of the ability of ATRA to reverse atrial fibrosis in more advanced stage HF.

3: Studies to define the ionic mechanisms by which ATRA improves atrial conduction parameters and studies of the impact on atrial arrhythmia susceptibility.

The present disclosure identifies ATRA as a novel therapy that can prevent and reverse atrial remodeling in cardiac pressure overload states. As there are currently no effective therapies that target the underlying molecular basis of atrial remodeling, this finding represents an important advance in the field of cardiac electrophysiology.

The present inventors study how cardiac pressure overload affects the expression of ATRA and how altered ATRA production or degradation contributes to pathological remodeling, as well as the underlying mechanisms by which ATRA confers protection against atrial conduction disease, inflammation, and fibrosis, the principal causes of atrial myopathy.

### Example 2. Left Atrial Expression of ALDHIA2 is Lower in Patients with Reduced LVEF, and CYP26B1 Expression is Increased in Patients with Persistent AF

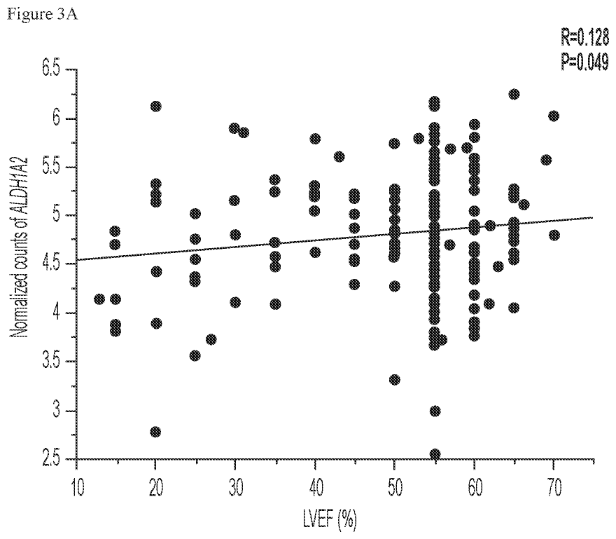

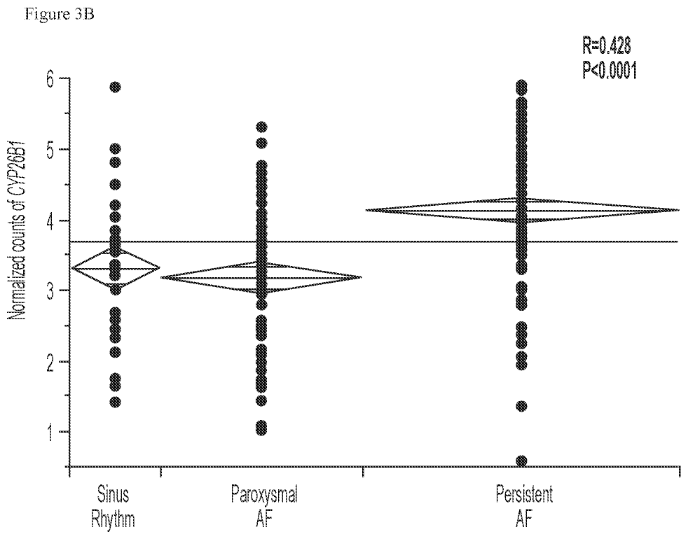

PolyA+RNA sequencing was performed on left atrial appendage (LAA) tissues from a total of 265 subjects (235 subjects of European descent, and 30 of African descent, mean age 60+12 years, 181 males). ALDH1A2 (ATRA biosynthesis) expression was lower in the LA of patients with reduced LVEF, indicating that atrial ATRA synthesis was reduced in HF patients (FIGS. 3A-3B). CYP26B1 (ATRA degradation) expression was higher in the LA of patients with persistent AF compared to sinus rhythm or paroxysmal AF patients, suggesting that increased degradation of endogenous ATRA may be involved in transitioning AF from paroxysmal to persistent form.

Summary: Reduced expression of left atrial ALDH1A2 in HF patients and increased expression of left atrial CYP26B1 in persistent AF patients suggest that reduced LA ATRA levels due to decreased biosynthesis or increased degradation may contribute to atrial remodeling that leads to AF.

### Example 3. ATRA Protects Against Pathological Atrial Remodeling in TAC Banded Mice

To define the role of ATRA in cardio-protection against pathological LA remodeling induced by pressure overload, the TAC banding model was used. TAC banding creates a fixed afterload obstruction in the systemic circulation and serves as an excellent model for drug testing as the surgically-induced coarctation cannot be reversed by medical therapy. It has been previously shown that TAC banding for two weeks causes left atrial electrical and structural remodeling in a highly reproducible manner before the development of overt HF.1 For the ATRA protection study, the study design consisted of C57B1/6 wildtype male and female mice divided evenly into four groups: i) Sham+V (Sham+vehicle control), ii) Sham+ATRA, iii) TAC+V (TAC+vehicle control), and iv) TAC+ATRA for a two-week study duration. ATRA (10 mg/kg/day, intraperitoneal (IP) injection) or vehicle control was first administered 30 minutes prior to TAC or Sham surgery and then daily for two weeks.

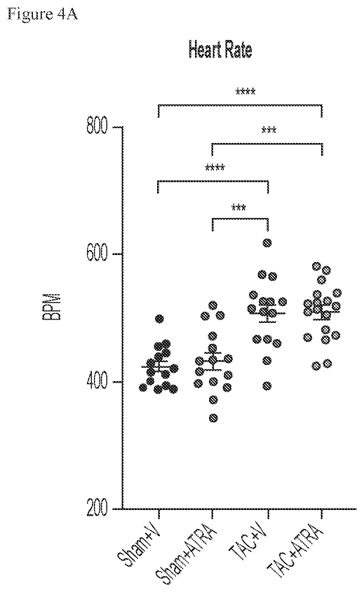

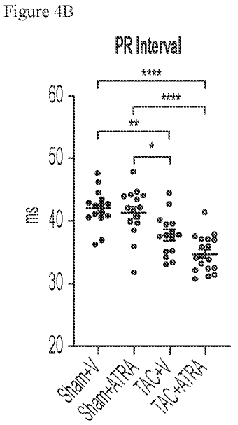

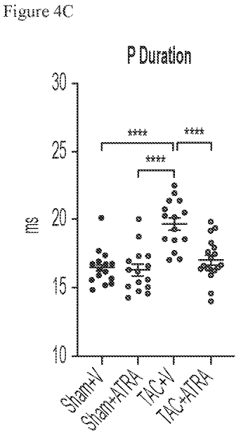

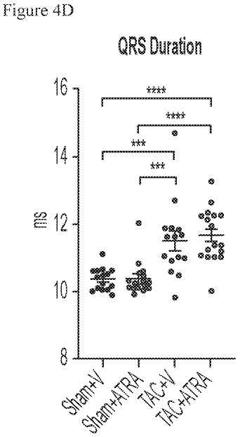

Electrocardiogramas performed on Sham+V, Sham+ATRA, TAC+V, and TAC+ATRA cohorts at two weeks to investigate the effect of ATRA treatment on atrial and ventricular activation and repolarization parameters. On ECG, the P wave duration is a measure of atrial activation time, the PR interval is a measure of atrial-to-ventricular activation time that includes the atrio-ventricular nodal conduction time, the QRS duration is a measure of ventricular activation time, and the HR-corrected QT (QTc) interval is a measure of ventricular repolarization time. There were no differences in ECG measurements in the Sham+V versus Sham+ATRA groups, indicating that ATRA treatment had no discernable effect on cardiac activation or repolarization parameters under normal pressure conditions (FIGS. 4A-4E). In TAC banded mice treated with vehicle control (TAC+V), the heart rate (HR) was increased, the P wave, QRS wave, and QTc interval durations were prolonged, and the PR interval was shortened compared to Sham groups. In TAC banded mice treated with ATRA (TAC+ATRA), the P wave duration was reduced to normal levels, whereas HR, PR interval, QRS wave, and QTc interval durations were unchanged compared to the TAC+V cohort.

Summary: ATRA normalizes atrial activation time in TAC banded mice, but had no effect on HR or ventricular activation and repolarization times. These findings indicate that despite the fixed afterload obstruction and evidence of ventricular electrical remodeling by ECG, ATRA has a profound protective effect on atrial electrical remodeling.

### Example 4. ATRA Improves Left Atrial Functional Properties in TAC Banded Mice

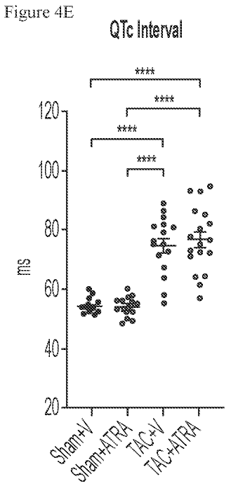

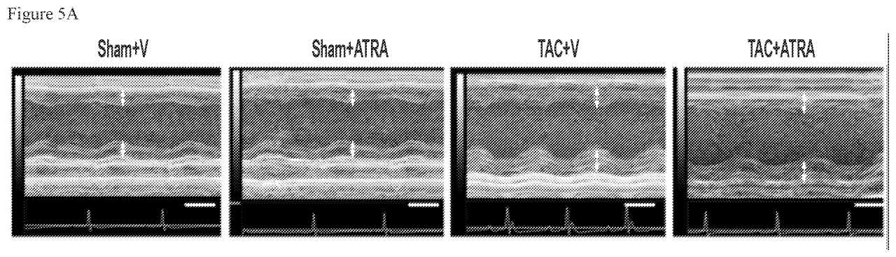

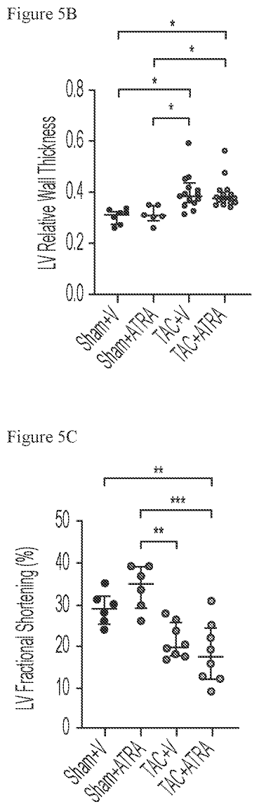

Transthoracic echocardiography (TTE) was performed on Sham+V, Sham+ATRA, TAC+V, and TAC+ATRA cohorts at two weeks to investigate the effect of ATRA treatment on atrial and ventricular structure and function. There were no differences in echocardiographic measurements of the LA and left ventricle (LV) in the Sham+V versus Sham+ATRA groups, indicating that ATRA treatment had no discernable effect on gross cardiac structure or function under normal pressure conditions (FIGS. 5A-5E). In the TAC+V and TAC+ATRA groups, there was a similar degree of left ventricular hypertrophy (LVH) and reduced left ventricular systolic function as measured by fractional shortening and longitudinal strain analysis compared to the Sham+V and Sham+ATRA groups (FIG. 5B-D). These findings indicate that ATRA did not affect the left ventricular hypertrophic response or the decline in left ventricular systolic function in response to pressure overload. The effect of TAC banding on left atrial function was measured next by using reservoir strain analysis. 8,30 Left atrial reservoir strain analysis is a sensitive modality to study left atrial mechanics of both relaxation (conduit strain) and contraction (contractile strain) in an angle-independent fashion. Left atrial mechanics in the TAC+ATRA group were significantly improved compared to the TAC+V group, although the values did not return to normal levels (FIG. 5E).

Summary: ATRA improves left atrial functional parameters but has no effect on LVH or left ventricular function. These findings indicate that despite the fixed afterload obstruction and ventricular structural remodeling, ATRA improves left atrial myocardial physiology.

### Example 5. ATRA normalizes left atrial conduction velocity (CV) in TAC banded mice

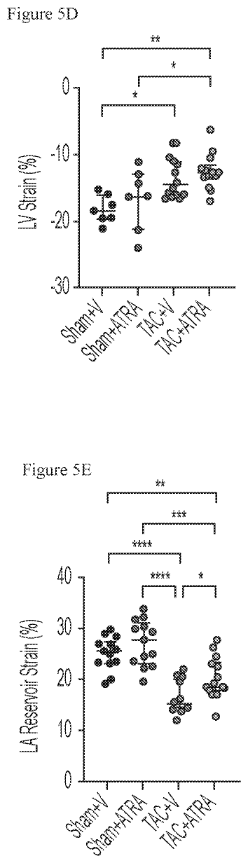

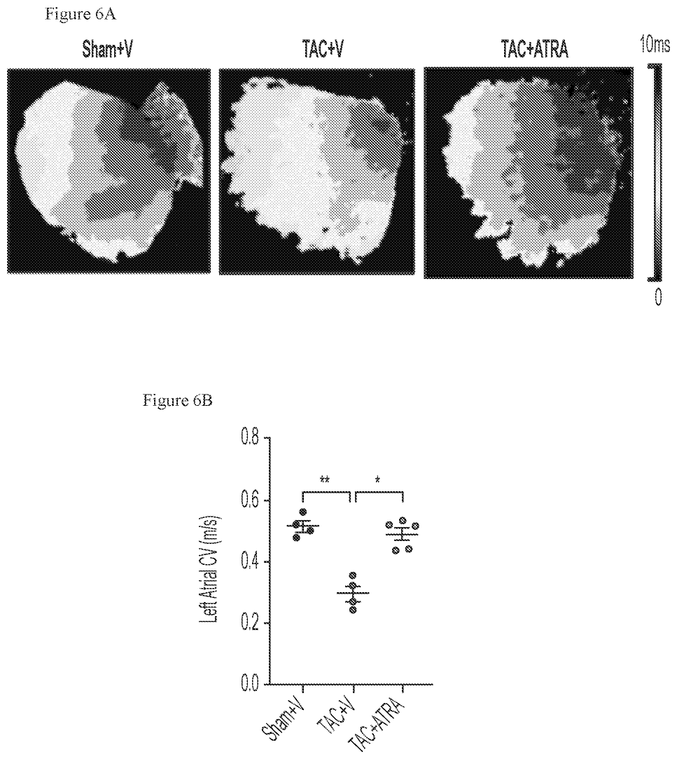

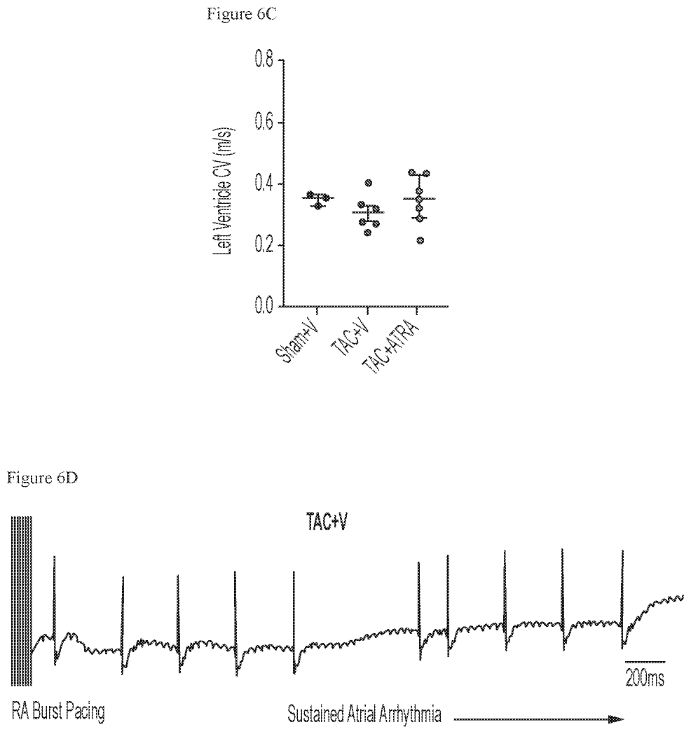

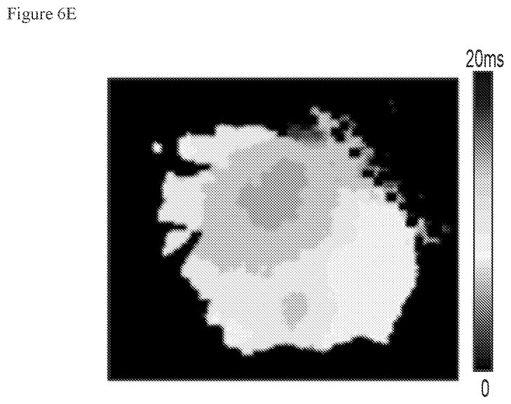

Optical mapping was performed next on Langendorff-perfused Sham+V, TAC+V, and TAC+ATRA hearts to assess left atrial and left ventricular conduction properties (FIGS. 6A-6E). Representative isochronal maps of Sham+V, TAC+V and TAC+ATRA left atria are shown in FIG. 6A. Consistent with P wave duration prolongation on ECG in the TAC+V group, left atrial CV was significantly reduced in TAC+V hearts compared to Sham+V hearts (FIGS. 6A, 6B). In the TAC+ATRA group, left atrial CV was significantly increased to normal levels (CV=0.52+0.02 m/s for Sham+V, 0.30=0.03 m/s for TAC+V and 0.50+0.02 m/s for TAC+ATRA, p<0.05, one-way ANOVA) (FIG. 6B). Left ventricular CVs in the Sham+V, TAC+V, and TAC+ATRA hearts were not significantly different between the three groups.

Atrial arrhythmia inducibility was assessed by performing atrial burst pacing protocols in TAC+V and TAC+ATRA hearts.⁶ Atrial burst pacing induced sustained atrial arrhythmia (lasting >30 sec) in 1 out of 3 TAC hearts. In contrast, 0 out of 3 TAC+ATRA hearts displayed sustained atrial arrhythmia. Induction of sustained atrial tachycardia in the TAC+V heart is shown in FIG. 6C. Optical map of the TAC+V LA during atrial tachycardia revealed abnormal activation wavefronts originating from the body of the LA (FIG. 6D).

Summary: ATRA normalizes left atrial CV in TAC banded hearts.

### Example 6. ATRA protects against left atrial fibrosis in TAC banded mice

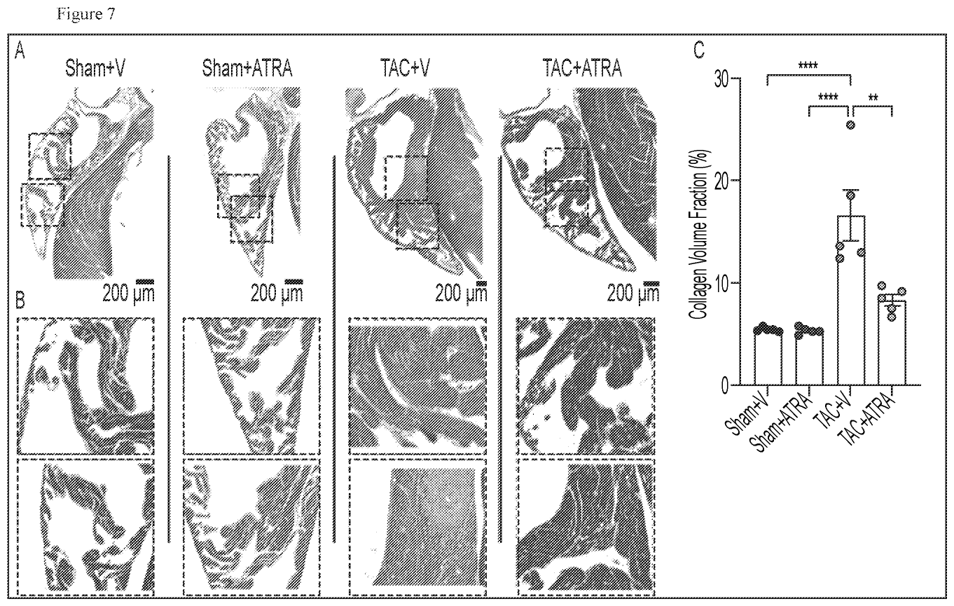

To investigate the mechanisms by which ATRA restores normal left atrial CV, changes in left atrial fibrosis burden were evaluated by using ImageJ analysis on Masson's trichrome stained sections, quantifying the entire LA region per section.1 As demonstrated in FIG. 7, there were no differences in fibrosis levels as measured by collagen volume fraction between Sham+V and Sham+ATRA samples, indicating that ATRA does not influence fibrosis under normal pressure conditions. Both the TAC+V and TAC+ATRA groups showed left atrial hypertrophy, consistent with elevated intracardiac pressure. In the TAC+V LA, there was significant increase in collagen volume fraction in the interstitium and in the perivascular regions compared to Sham+V and Sham+ATRA groups. Notably, TAC+ATRA LA showed significant reduction in collagen volume fraction with reduced left atrial interstitial and perivascular fibrosis compared to TAC+V LA. Collagen volume fraction in the TAC+ATRA LA was not significantly different from Sham groups (5.4±0.1% for Sham+V, 5.3±0.1% for Sham+ATRA, 16.6±2.5% for TAC+V, and 8.3±0.6% for TAC+ATRA).

Summary: ATRA protects against atrial fibrosis in the pressure overloaded heart.

### Example 7. ATRA Prevents Cx43 Remodeling in the LA of TAC Banded Hearts

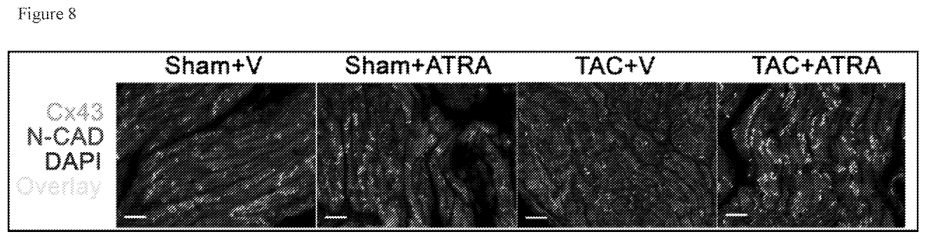

As Cx43 remodeling is a known contributor to conduction disease in the pressure overloaded heart, it was investigated whether ATRA treatment protects against Cx43 gap junction remodeling using immunofluorescence staining in Sham+V, Sham+ATRA, TAC+V, and TAC+ATRA LA sections. Sham+V and Sham+ATRA LA showed similar levels of Cx43 expression and proper localization at the intercalated discs, as evidenced by co-immunostaining with N-cadherin (N-CAD) (FIG. 8). In the TAC+V LA, Cx43 expression appeared reduced and was relocalized to the lateral membranes (i.e. gap junction remodeling). In the TAC+ATRA LA, Cx43 expression was robust and was properly localized to the intercalated discs.

Summary: ATRA treatment prevents Cx43 remodeling in the pressure overloaded heart.

### Example 8. ATRA Regulates a Transcriptional Program in the Pressure Overloaded LA that Protects Against Electrical and Structural Remodeling

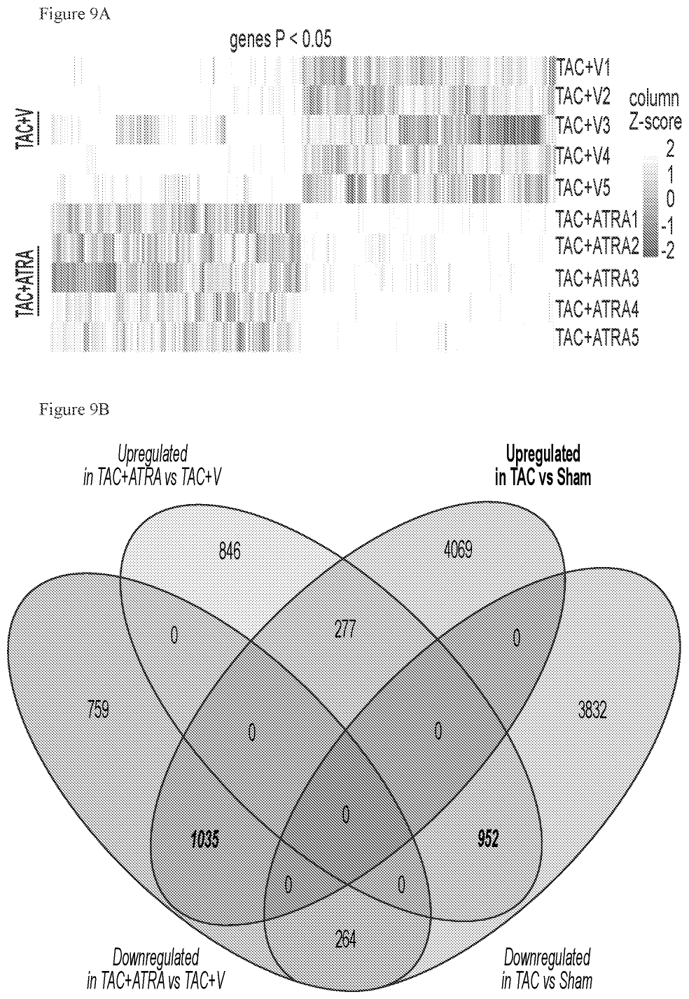

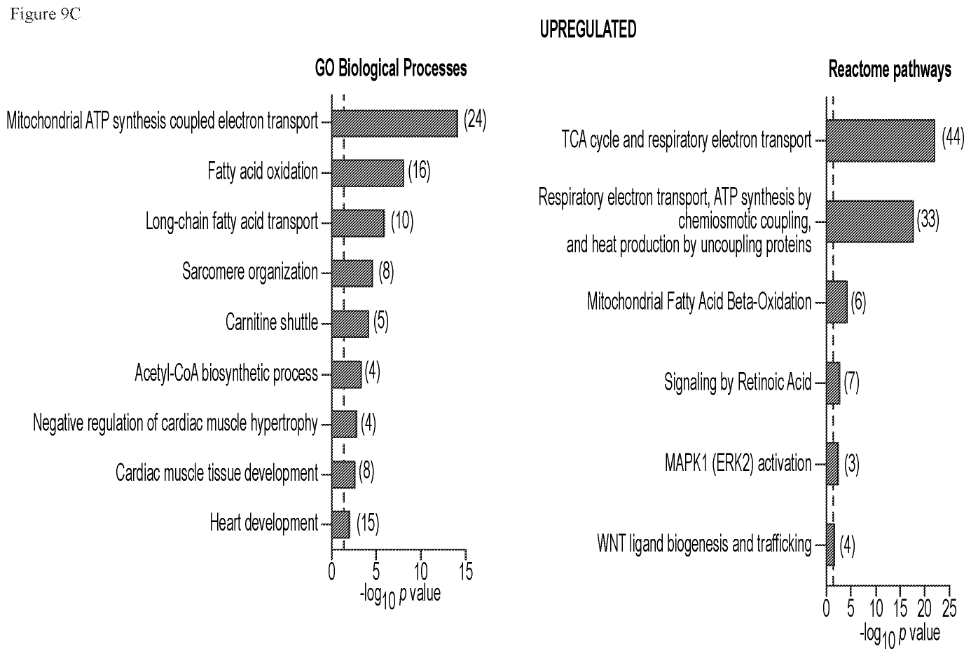

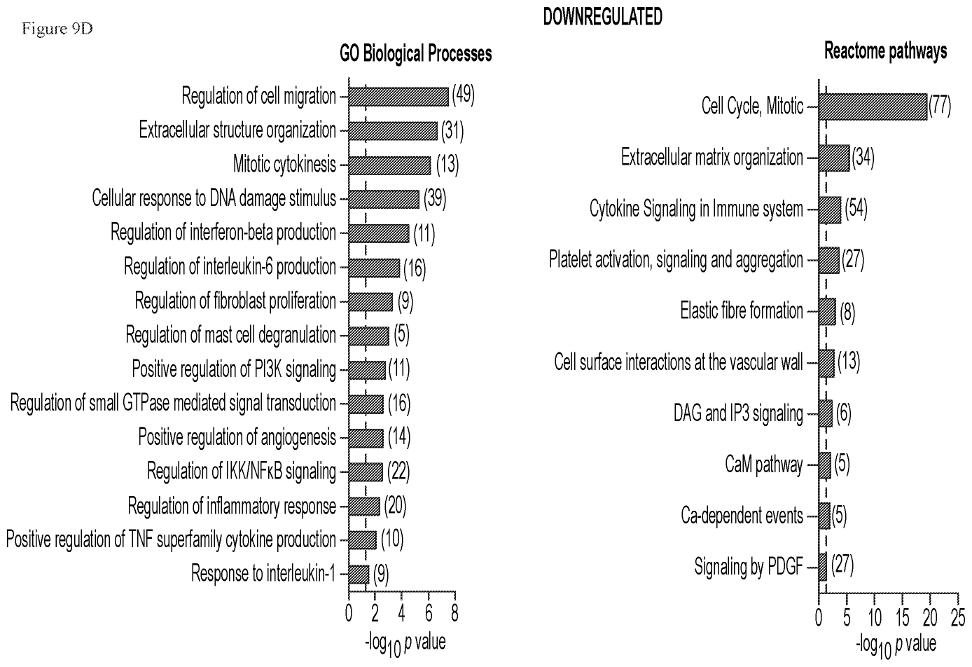

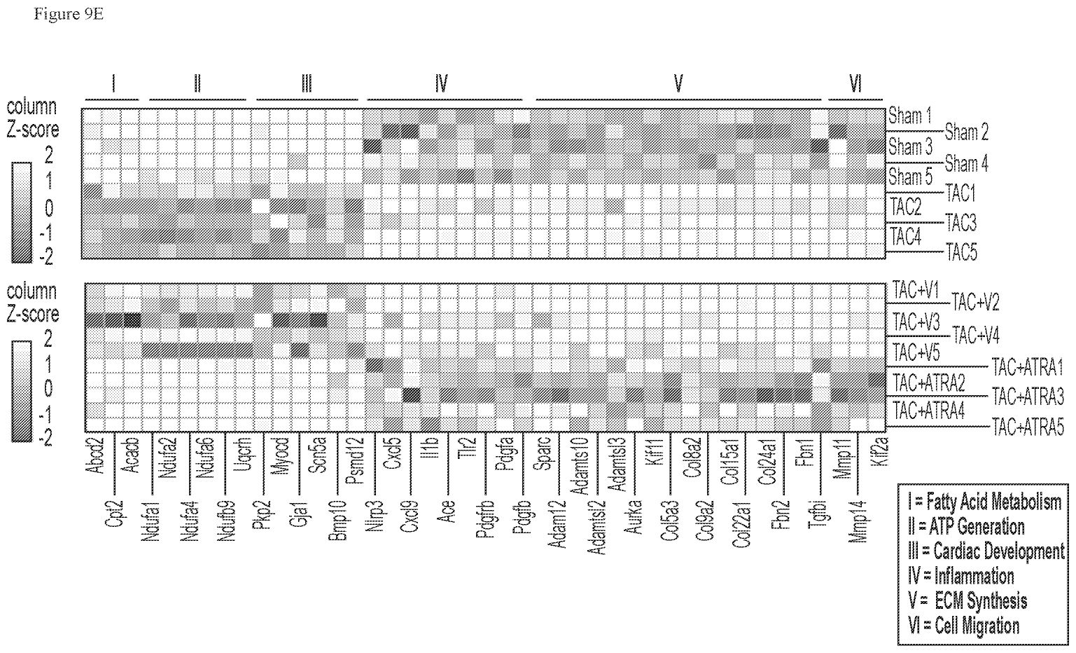

To define the ATRA-dependent transcriptional program in the LA that protects against pressure-induced remodeling, RNA-seq was performed on LA samples from TAC+V and TAC+ATRA hearts (FIGS. 9A-9E). Principal component analysis (PCA) and Euclidean plots of global gene expression data illustrated clear separation of TAC+V and TAC+ATRA samples. Using a threshold criterion of P<0.05 resulted in 4133 transcripts that were differentially expressed (2075 transcripts upregulated and 2058 transcripts downregulated) between TAC+ATRA versus TAC+V LA samples. Heatmap analysis of the 4133 genes showed excellent segregation of gene expression by treatment groups (FIG. 9A). Comparative analysis of differentially expressed genes (DEGs) from the [TAC+ATRA vs TAC+V] dataset compared against the [TAC vs Sham] dataset was then performed to generate the Venn diagram in FIG. 9B. Based on the Venn diagram analysis, 1987 DEGs (952 upregulated and 1035 downregulated genes) were identified in the [TAC+ATRA vs TAC+V] dataset that move in reciprocal direction to the [TAC vs Sham] dataset, representing “normalization” of adversely remodeled genes.

Gene ontology (GO) biological process and REACTOME pathway analyses were then performed using Enrichr on significantly upregulated and downregulated genes that were “normalized” with ATRA treatment (FIGS. 9C, 9D). Major biological processes that are significantly upregulated with ATRA treatment include pathways involved in mitochondrial energy production [mitochondrial ATP synthesis coupled electron transport, fatty acid oxidation] and signaling pathways involved in cardiac/atrial development [WNT ligand biogenesis and trafficking, signaling by retinoic acid, sarcomere organization, heart development]. Major biological processes and pathways that are significantly downregulated with ATRA treatment include regulation of ECM production [extracellular structure organization, ECM organization], pro-inflammatory signaling [response to interleukin-1 (IL-1), regulation of interleukin-6 (IL-6) production, positive regulation of TNF superfamily cytokine production, regulation of inflammatory response, regulation of I-kB kinase/NF-KB signaling, cytokine signaling in immune system, regulation of mast cell degranulation, signaling by PDGF], and regulation of vasoconstriction [regulation of systemic arterial blood pressure]. Representative DEG's in TAC banded LA (upper panel) that are “normalized” by ATRA therapy (bottom panel) are categorized by biological processes in FIG. 9E.

### Example 9. ATRA Therapy Downregulates Key Inflammatory Signaling Pathways in the Pressure-Overloaded LA

The present pathway analysis showed that ATRA therapy downregulates 3 important mechanisms of inflammation in the pressure overloaded LA: i) the TLR/NF-KB/NLRP3 signaling pathway for pro-inflammatory cytokine (IL-1b) production and activation, ii) mast cell-dependent PDGF-A activation, and iii) AngII signaling activation. ATRA downregulated the expression of Toll like receptor 2 (TLR2), NF-KB signaling components, NLRP3 (NACHT, LRR, and PYD domain containing protein 3), and IL-1b (FIGS. 9D, 9E). Importantly, TLR2, NF-KB, NLRP3, and IL-1b have all been shown to be upregulated in AF patients.31-35 In diabetic nephropathy, ATRA has been shown to block cytokine, chemokine, and growth factor production through inhibition of TLR/NF-KB signaling.28 In addition, TLR236 and TLR 428,34 transcriptionally regulate the expression of the NLRP3 inflammasome, which converts pro-inflammatory cytokines to activated forms, such as pro-IL-1b to IL-1b, via activated-caspase-I (Casp1-p20 and Casp1-p10) dependent cleavage.37 Another signaling pathway that has been shown to play a significant role in atrial fibrosis during cardiac pressure overload is through mast cell-dependent PDGF-A secretion.⁶ PDGF-A was shown to activate cell proliferation and collagen synthesis in cardiac fibroblasts.⁶ The present pathway analysis shows that ATRA treatment downregulates mast cell degranulation and PDGF signaling pathways, with reduced expression of PDGF-A and PDGF-B (FIGS. 9D, 9E). Lastly, it is notable that the angiotensin converting enzyme (ACE), which is upregulated with TAC banding, was significantly downregulated with ATRA therapy (FIGS. 9D, 9E). Activation of the AngII signaling pathway in the LA from pressure overload is a powerful inducer of inflammation, fibrosis, and Cx43 gap junction remodeling. Blockade of AngII signaling can reduce atrial fibrosis and partially rescued Cx43 remodeling. ^{38,39}

Summary: ATRA downregulates key inflammatory pathways implicated in atrial fibrosis and electrical remodeling.

### Example 10. Delayed ATRA Treatment Reverses Pathological Atrial Remodeling at Later Stages of Cardiac Pressure Overload

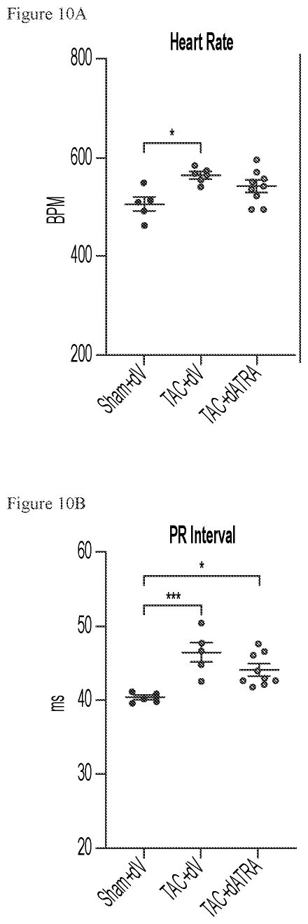

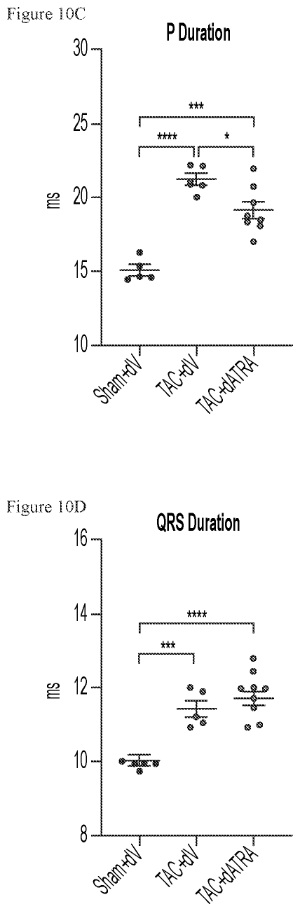

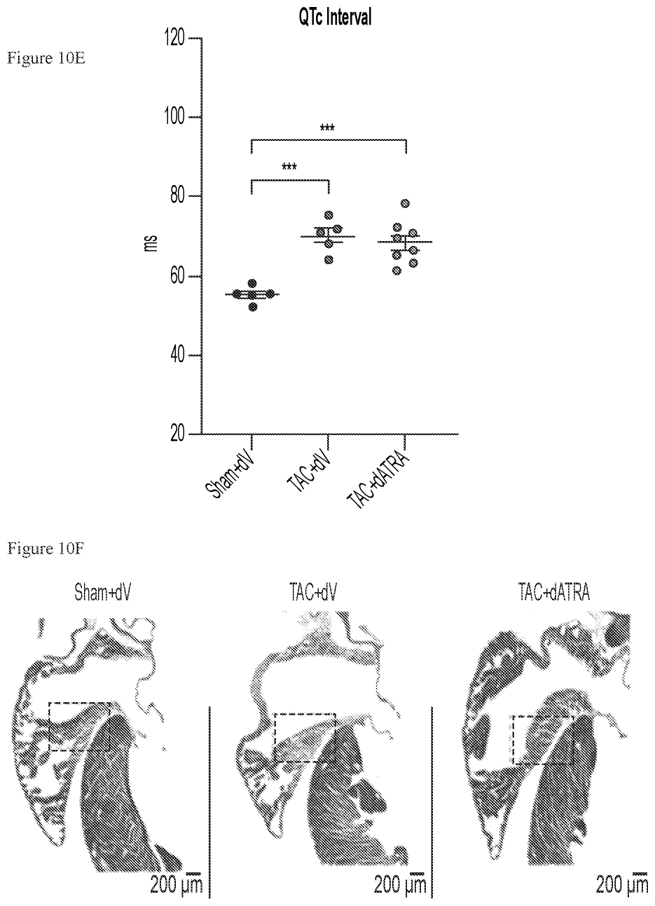

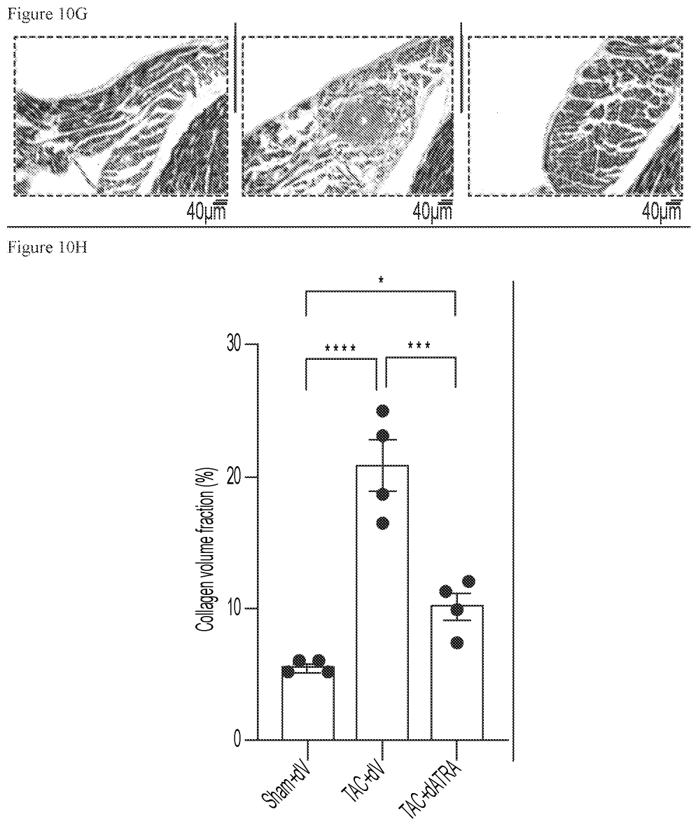

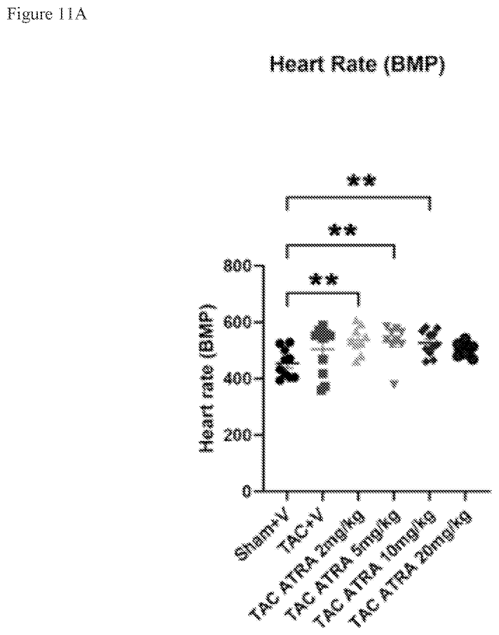

To investigate the ability of ATRA to reverse atrial remodeling, a new study was designed in which ATRA therapy would be initiated two weeks after TAC or Sham surgery. This model more accurately reflects the clinical presentation of HF patients. For the ATRA reversal study, C57B1/6 wildtype male and female mice were divided evenly into three groups: i) Sham+dV (delayed vehicle initiation), ii) TAC+dV, and iii) TAC+dATRA (delayed ATRA initiation). Mice underwent Sham or TAC surgery and were left untreated for two weeks. ATRA (10 mg/kg/day, IP injection) or vehicle control was started at two weeks post-TAC or Sham surgery and continued for two weeks, making the total study duration four weeks. At the end of the 4-week study, ECG and histological analyses were performed. In the TAC+dV cohort, there was an increase in HR and prolongation of the P wave duration, PR interval, QRS wave duration, and QTc interval by ECG (FIGS. 10A-E). In the TAC+dATRA group, P wave duration was significantly reduced compared to the TAC+dV group, whereas the PR interval, QRS duration, and QTc interval were not significantly different compared to the TAC+dV group.

LA fibrosis burden was then quantified using ImageJ analysis on Masson's trichrome stained sections (FIGS. 10F-H). The TAC+dV LA at 4 weeks showed progressive fibrosis accrual with collagen volume fraction measured at 21.9+1.8% compared to 16.6+2.5% for the TAC+V LA at two weeks (FIG. 10H and FIG. 7C). Remarkably, delayed ATRA treatment in the TAC+dATRA LA reduced collagen volume fraction to 10.9+1.1% at 4 weeks, which is below expected levels measured in TAC+V LA at two weeks (FIG. 10H and FIG. 7C). These data indicate that delayed ATRA therapy can reverse the fibrotic process in the LA of pressure overloaded hearts.

Summary: ATRA treatment reduces atrial electrical remodeling and reverses atrial fibrosis due to cardiac pressure overload.

### Example 11. ATRA Treatment Demonstrates a Dose-Response Curve in Normalizing Atrial Electrocardiogrameters in TAC Banded Mice

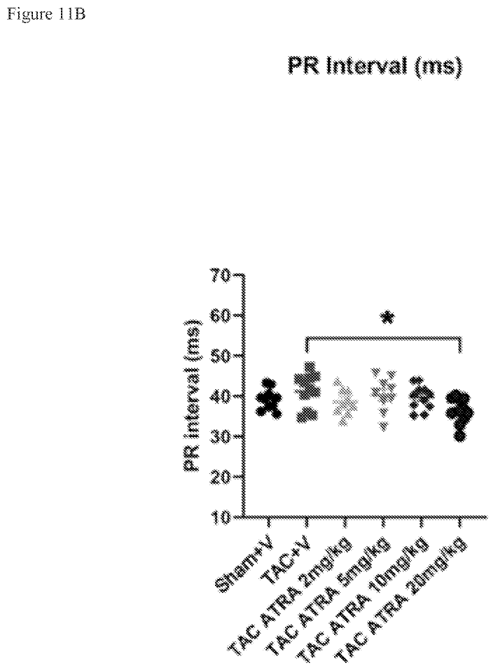

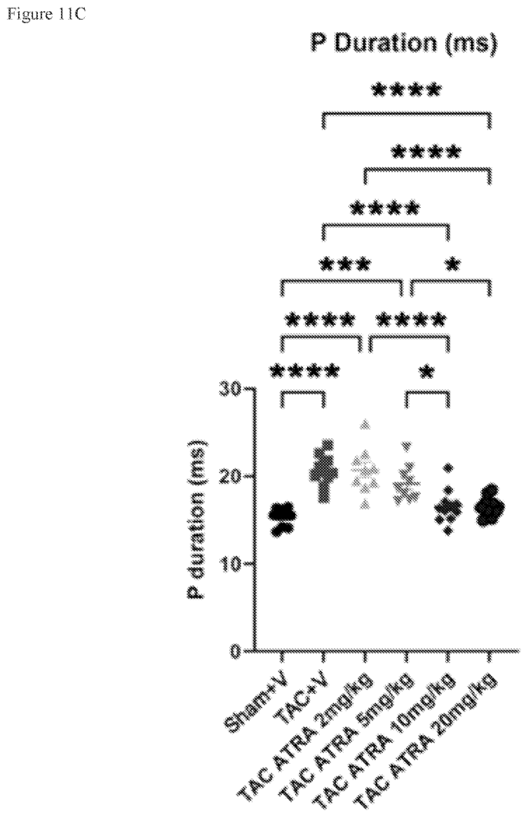

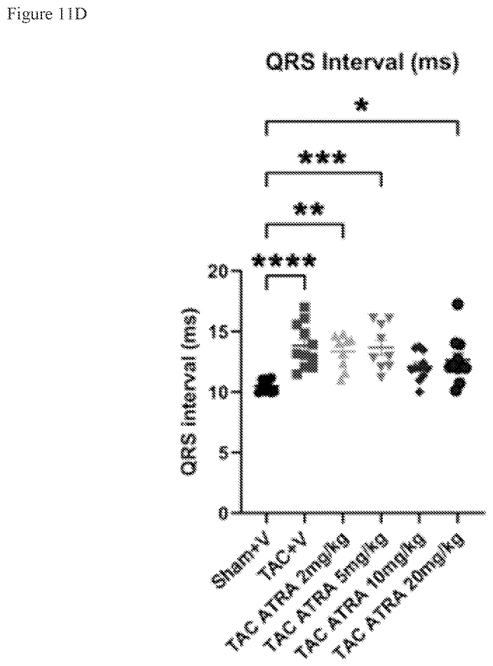

To investigate the dose-dependent effect of ATRA on atrial conduction parameters, electrocardiogramas performed on Sham+V, TAC+V, and TAC+ATRA cohorts at two weeks using various doses of ATRA (2 mg/kg, 5 mg/kg, 10 mg/kg, and 20 mg/kg administered daily via intraperitoneal injection). Although there was no significant dose-dependent effect of ATRA on heart rate, PR interval or QRS duration, ATRA treatment reduced P wave prolongation in a dose-dependent fashion (FIGS. 11A-11D). Maximal P wave duration shortening was seen at an ATRA dose of 10m/kg, with no further shortening with the 20 mg/kg dose. Notably, ATRA 20 mg/kg demonstrated increased ventricular ectopy, indicating potential cardiotoxicity at this high dose.

Summary: ATRA normalizes atrial activation time in TAC banded mice in a dose-dependent fashion.

### Example 12. Additional Experiments

1: Study the role of endogenous ATRA in maintaining normal atrial physiology, the impact of pressure overload on endogenous ATRA levels, and the therapeutic potential of raising endogenous ATRA levels to prevent TAC-induced LA remodeling.

Hypothesis: Endogenous ATRA is necessary to maintain normal atrial physiology, TAC banding reduces LA and blood ATRA levels, and inhibition of ATRA degradation pathways restores LA ATRA levels and prevents atrial remodeling.

1.1 Investigation of the maintenance role of ATRA in atrial electrical and structural homeostasis. The present data showed enrichment of Aldh1a1 and Aldh1a2 expression and relative deficiency of Cyp26b1 expression in postnatal atrial myocytes compared to ventricular myocytes, indicating that ATRA signaling remains enriched in the postnatal atrium. It is hypothesized herein that ATRA, which has an essential role in atrial specification, continues to have a maintenance role in atrial electrical and structural integrity in the adult heart. Differential expression of ALDHA1A1, ALDHA1A2, and CYP26B1 at the protein level in the atria versus ventricles of the adult heart will be confirmed using western blot and immunofluorescence staining. ATRA levels in the atria, ventricles, and blood plasma will then be quantified. Quantification of endogenous ATRA levels will be performed. To investigate the role of ATRA in maintaining adult atrial electrical and structural integrity, wildtype C57B1/6 male and female mice will be treated with the ALDHIA1 and ALDH1A2 specific inhibitor WIN18446 (2 mg/g of diet) for a total of 4 weeks, which has previously been shown to efficiently block ATRA biosynthesis in vivo.⁴⁰ ATRA levels in the atria, ventricles, and plasma will be quantified after WIN18446 treatment to ensure endogenous suppression. We will assess the impact of ATRA depletion on ECG and TTE parameters, the degree of fibrosis within the atria and ventricles will be quantified using Masson's trichrome staining. Based on electrophysiological or fibrotic changes in the LA, optical mapping will be performed to assess conduction and repolarization parameters. Differential gene expression of ion channels or pro-fibrotic pathways will be also assessed as performed in the TAC+ATRA experiments.

1.2 Study of the effect of cardiac pressure overload on endogenous ATRA levels in the LA and in the plasma. It has been reported that ATRA levels are reduced in failing ventricular myocardium by mass spectrometry in patients with idiopathic dilated cardiomyopathy (IDCM) and in a guinea pig HF model.26 ATRA levels will be quantified in the LA and in blood plasma of Sham and TAC banded mice in the NYU Metabolomics Core Resource Laboratory, as described in section 1.1, above. Paired analysis of ATRA levels from plasma and LA tissue will be performed to assess whether plasma ATRA levels correlate with LA tissue ATRA levels. This data will provide information as to whether plasma ATRA levels are a good surrogate measure of LA ATRA levels. The relationship between atrial and plasma ATRA levels and the degree of atrial electrical and structural remodeling will be also studied.

1.3 Study of the effect of the CYP26 inhibitor talarozole in preventing atrial electrical and structural remodeling with cardiac pressure overload. Left atrial CYP26B1 transcript levels were increased in patients with persistent AF. Similarly, Cyp26b1, as well as Cyp26a1 and Cyp26cl, were significantly upregulated in the LA of TAC banded hearts at two weeks. These data suggest that increased ATRA degradation may be a cause of ATRA deficiency in the LA in humans and mice, contributing to atrial remodeling. To test this hypothesis, all CYP26 isoforms will be blocked using pan-CYP26 inhibitor talarozole that has been shown to effectively increase ATRA levels in rodents.⁴¹ The first dose of talarozole (2.5 mg/kg) or vehicle control will be administered 12 hours before TAC banding and administer twice daily for two weeks, based on previously published data.⁴¹ After two weeks post-TAC, physiological testing will be performed using ECG and TTE. Histological analysis of the LA will also be performed to quantify fibrosis burden. If talarozole has a positive influence on atrial conduction parameters, optical mapping will be performed to measure conduction and repolarization parameters. Talarozole effect on ATRA levels will also be performed in the LA, as described in 1.1 and 1.2.

Discussion: Based on the present findings, inhibition of endogenous ATRA is expected to have a deleterious effect on atrial conduction parameters and result in activation of pro-inflammatory and pro-fibrotic pathways, albeit at a lower severity than with TAC banding. By concomitantly measuring plasma and left atrial ATRA levels after TAC banding, it will be determined whether plasma ATRA levels are a good surrogate measure of left atrial ATRA levels. It will also be determined whether plasma and left atrial ATRA levels directly correlate with the severity of atrial conduction disease and fibrosis. This will yield information guiding potential therapy in patients with cardiac pressure overload conditions. Lastly, it will be determined whether CYP26B1 inhibition can raise left atrial ATRA levels to a therapeutic threshold that can prevent pressure-induced remodeling. There is a possibility that measured left atrial ATRA levels will be unchanged or higher with TAC banding. However, the present data showing a beneficial response with ATRA supplementation therapy indicates that irrespective of the endogenous ATRA levels reached, they are insufficient to prevent adverse remodeling with pressure overload.

2: Define the anti-inflammatory mechanisms by which ATRA prevents and reverses atrial fibrosis and investigate the ability of ATRA to reverse atrial fibrosis in more advanced stage HF.

Hypothesis: ATRA therapy prevents and reverses atrial fibrosis by negatively regulating pro-inflammatory cytokine/chemokine/growth factor pathways both in early and more advanced-stage HF.

2.1 Study of the impact of ATRA treatment on proinflammatory cytokines, chemokines, and mediators in the pressure-overloaded LA. The present RNA-seq analysis indicates that ATRA reduces fibrosis by decreasing the expression of pro-inflammatory cytokines, chemokines, cell adhesion molecules, and growth factors. To define the key inflammatory pathways regulated by ATRA in the pressure overloaded LA, antibody array kit panels (RayBio® C-Series Mouse Inflammation Antibody Array 1 Kit and the Mouse Growth Factor Array C3) will be used to test the expression of 60 pro-inflammatory cytokines, chemokines, and growth factors at the protein level. This unbiased protein level screen will be used to identify the pro-inflammatory cascades most prominently affected by ATRA treatment and correlate this data with the inflammatory pathways identified by our comparative RNA-seq analysis.

2.2 Study of the impact of ATRA treatment on mast cell-dependent PDGF signaling.

To assess the effect of ATRA treatment on mast cell recruitment and activation in the LA of TAC banded hearts, staining with toluidine blue (0.1%; Sigma-Aldrich) and avidin conjugated to fluorochrome dyes (rhodamine labelled-avidin, 1:100; Vector Laboratories) will be performed to quantify the number and activity level of mast cells in the LA on histological sections.⁶ Avidin conjugated to fluorochrome dyes binds to the negatively charged heparin proteoglycans and identifies mast cells that have degranulated. ATRA effect on PDGF-A and PDGF-B levels will be assayed using the growth factor array data in 2.1. Knowledge gained from this study will provide information on how ATRA prevents the recruitment or activation of mast cells in the pressure overloaded LA.

2.3 Study of the impact of ATRA treatment on the TLR2/NF-KB/II-1b/NLRP3 inflammasome pathway. The effect of ATRA treatment on the expression levels of TLRs (TLR2 and TLR4), NLRP3, and activated caspase-1 (Casp1-p20) in Sham+V, TAC+V, and TAC+ATRA will be studied by Western blot. Changes in IL-1b expression will be assayed using the inflammation array kit used in 2.1. The effect of ATRA on NF-KB activation status will also be studied by: 1) quantifying protein levels of the NF-KB inhibitor, IkB-a, 2) quantifying the phosphorylation (activated) state of NF-KB/p65, and 3) quantifying the nuclear localization status of NF-KB/p65. This will allow to determine the effects of ATRA on the TLR-dependent signaling pathway, which serves as a regulatory hub for the NLRP3 inflammasome and a production hub for pro-inflammatory cytokines, chemokines, and growth factors.

2.4 Study of the effects of ATRA treatment on the renin-angiotensin system (RAS) signaling pathway. Activation of the RAS signaling pathway in the LA from pressure overload is a powerful inducer of atrial inflammation, fibrosis, and electrical remodeling. AngII promotes inflammation by increasing the production of pro-inflammatory cytokines, such as IL-6, TNF, and chemotactic signals that recruit immune cell migration. The angiotensin converting enzyme (ACE), which is upregulated with TAC banding, is significantly downregulated with ATRA therapy, providing an additional mechanism for reduced inflammation. The effects of ATRA treatment on ACE expression will be validated at the protein level. The effects of ATRA treatment on circulating AngII levels in the serum of Sham, Sham+ATRA, TAC, and TAC+ATRA mice will also be studied by using an AngII Enzyme Immunoassay Kit (RAB0010, Sigma-Aldrich). The effects of ATRA therapy on IL-6 and TNF expression will also be studied by using the inflammation array kit from 2.1.

2.5 Investigation of the ability of ATRA to reverse atrial fibrosis in more advanced stages of HF. As the delayed ATRA initiation model more accurately reflects the clinical presentation of HF, the effects of ATRA on atrial remodeling when initiated at progressively later time points will be investigated using TAC banding. The present data indicate that ATRA can reverse atrial fibrosis when initiated two weeks after TAC banding. To study if ATRA can reverse fibrosis at even later time points after TAC banding, ATRA therapy or vehicle control will be initiated after four-or six-weeks post-TAC banding and continue therapy for two weeks. The effects of ATRA on preventing progression or reversing atrial remodeling will be investigated initially with ECG and TTE. Fibrosis in the LA will be quantified using ImageJ analysis of Masson's trichrome stained sections. Optical mapping will also be performed to quantify changes in atrial and ventricular conduction velocity and repolarization parameters.

Discussion: Experiments described above will explore the mechanisms underlying the powerful anti-inflammatory and anti-fibrotic effects of ATRA in the pressure-overloaded LA.

3: Define the ionic mechanisms by which ATRA improves atrial conduction parameters and investigate the impact on atrial arrhythmia susceptibility.

Hypothesis: ATRA therapy improves atrial conduction parameters in part by normalizing sodium and gap junctional conductances. Improvements in atrial conduction parameters with ATRA treatment will reduce susceptibility to atrial arrhythmia.

3A.1 Quantification of Nav1.5 expression in the LA with ATRA treatment of TAC banded hearts. Scn5a expression is reduced at the RNA and protein levels in the LA after TAC banding for two weeks.1 ATRA treatment of TAC banded hearts increases left atrial Scn5a expression and improves conduction parameters. Western blot analysis will be performed to quantify Nav1.5 levels in the LA of TAC+ATRA and TAC+V groups. It will also be evaluated whether Nav1.5 is properly localized to the membrane with ATRA treatment using immunofluorescence staining.

3A.2 Patch clamp assay to assess the effect of ATRA treatment on sodium currents in TAC banded left atrial myocytes. Comprehensive biophysical evaluation of sodium currents in TAC+ATRA and TAC+V left atrial myocytes will be performed. 1,18,42

3A.3 Investigation of the effect of ATRA on the ErbB4 signal transduction pathway, a regulator of Scn5a expression. ErbB4 signal transduction pathway which enhances Scn5a expression in the atrial myocardium, is downregulated in the LA with TAC banding.1 Preliminary data obtained herein shows that ATRA treatment prevents the downregulation of ErbB4 in the LA with TAC banding at the protein level. The activation state of ErbB4 (phospho-ErbB4-Y1284) and its heterdimerzation partner ErbB2 (phospho-ErbB2-Tyr1248) will be investigated in the LA of TAC+ATRA and TAC+V hearts. Expression and activity levels of downstream effectors of ErbB4 signaling, such as MAPK ERK1/2 and transcription factor ETV1 will also be studied.^{43,44}

3B.1 Study of the mechanism of ATRA-dependent correction of Cx43 remodeling in the LA. The present data indicated that ATRA treatment prevents Cx43 remodeling in the LA. Phosphorylation of key serine residues (S325/S328/S330) of Cx43 (pS-Cx43) by the Ca2+/calmodulin protein kinase II (CaMKII) and casein kinase 1d (CK1d) have been shown to be important for assembly into gap junctions and maintenance at the intercalated disc.^{45,46} It is hypothesized herein that ATRA therapy prevents Cx43 remodeling by restoring phosphorylation of Cx43 at S325/S328/S330 sites. The percentage of total Cx43 phosphorylated at S325/S328/S330 will be quantified in LA samples from Sham, Sham+ATRA, TAC, and TAC+ATRA hearts using antibodies.

3B.2 Study of the role of ATRA in ameliorating CaMKII oxidation state and ROS production to protect against Cx43 remodeling. Oxidized CaMKII plays a significant role in Cx43 hypo-phosphorylation and remodeling.⁴⁶ It is hypothesized herein that ATRA treatment protects the LA against Cx43 remodeling in part by reducing oxidized CaMKII levels. Oxidized CaMKII levels will be quantified in Sham+V, Sham+ATRA, TAC+V, TAC+ATRA left atria using western blot analysis with antibodies directed against oxidized-CaMKII (Met281/282, MilliporeSigma, 07-1387; rabbit). TAC banding is a known inducer of reactive oxygen species (ROS) production. To assess whether ATRA treatment reduces ROS activation, isolated left atrial myocytes from Sham+V, Sham+ATRA, TAC+V, TAC+ATRA hearts loaded with a fluorescent ROS sensor, 2′,7′-dichlorodihydrofluorescein diacetate (H2DCFDA) will be studied. ^{46 Mitochondrial ROS production will also be measured using MitoSOX fluorescence using flow cytometry.}^{47,48}

3B.3 Study of the effects of ATRA treatment on CK1d protein stabilization. Although the present RNA-seq analysis did not show significant changes in CK1d transcript abundance with ATRA treatment, this does not rule out changes at the protein level. CK1d protein levels will be quantified in Sham, Sham+ATRA, TAC, TAC+ATRA left atria using western blot analysis.

3B.⁴ Study of the effects of ATRA treatment on the renin-angiotensin system (RAS) signaling pathway on Cx43 remodeling. Activation of the RAS signaling pathway in the LA from pressure overload is a powerful inducer of Cx43 gap junction remodeling. Blockade of the RAS pathway by inhibiting AngII synthesis (angiotensin-converting enzyme inhibitor, ACEI) or by directly blocking the angiotensin II receptor (angiotensin II receptor blocker, ARB) significantly reduced Cx43 remodeling. The effects of ATRA treatment on ACE expression at the protein level and AngII production will be explored, as described in 2.5.

3C Evaluation of the effect of ATRA therapy on arrhythmia susceptibility in TAC banded hearts. Ambient arrhythmia burden in the atrium and ventricles of TAC banded hearts treated with ATRA or vehicle control will be quantified by using implantable telemetry49. A comprehensive electrophysiology study will be performed to assess for baseline electrophysiological properties and to assess for arrhythmia susceptibility using provocative testing for atrial arrhythmias in vivo.⁴⁴ The optical mapping experiments will be completed by studying the effects of ATRA therapy on conduction parameters, repolarization properties, and arrhythmia susceptibility using programmed stimulation protocols on Langendorff-perfused hearts. 18,42,50

Discussion: Based on the present findings, it is predicted herein that ATRA will normalize sodium channel behavior and restore a signal transduction environment that prevents gap junction remodeling in the pressure overloaded heart. It will be determined whether ATRA restores pS-Cx43 levels and reduces oxidized-CaMKII levels by decreasing ROS activation. It will be also determined if ATRA stabilizes CK1d protein levels. It is expected that ATRA therapy will reduce RAS activation by decreasing ACE levels in the pressure overloaded LA. Knowledge gained from this study will provide mechanistic insight into how ATRA treatment corrects left atrial electrical remodeling.

Summary: The present study will address how ATRA regulates key inflammatory and ionic pathways to maintain normal adult atrial physiology as well as for therapeutic benefit to prevent and reverse pathological atrial remodeling that gives rise to AF. As there currently are no effective therapies that directly target atrial remodeling, this discovery is an important advance with high therapeutic potential.

### REFERENCES

1. Yamaguchi, N., et al. Cardiac Pressure Overload Decreases ETV1 Expression in the Left Atrium, Contributing to Atrial Electrical and Structural Remodeling. Circulation 143, 805-820 (2021).
2. Morillo, C.A., Banerjee, A., Perel, P., Wood, D. & Jouven, X. Atrial fibrillation: the current epidemic. J Geriatr Cardiol 14, 195-203 (2017).
3. January, C.T., et al. 2014 AHA/ACC/HRS guideline for the management of patients with atrial fibrillation: a report of the American College of Cardiology/American Heart Association Task Force on Practice Guidelines and the Heart Rhythm Society. J Am Coll Cardiol 64, el-76 (2014).
4. Marrouche, N. F., et al. Catheter Ablation for Atrial Fibrillation with Heart Failure. N Engl J Med 378, 417-427 (2018).
5. Bhat, A., et al. Drivers of hospitalization in atrial fibrillation: A contemporary review. Heart Rhythm 17, 1991-1999 (2020).
6. Liao, C. H., et al. Cardiac mast cells cause atrial fibrillation through PDGF-A-mediated fibrosis in pressure-overloaded mouse hearts. J Clin Invest 120, 242-253 (2010).
7. Wolowacz, S.E., Samuel, M., Brennan, V. K., Jasso-Mosqueda, J. G. & Van Gelder, I. C. The cost of illness of atrial fibrillation: a systematic review of the recent literature. Europace 13, 1375-1385 (2011).
8. Barbier, P., Solomon, S. B., Schiller, N. B. & Glantz, S. A. Left atrial relaxation and left ventricular systolic function determine left atrial reservoir function. Circulation 100, 427-436 (1999).
9. Singh, B. N., et al. Amiodarone versus sotalol for atrial fibrillation. N Engl J Med 352, 1861-1872 (2005).
10. Kochiadakis, G. E., et al. Amiodarone, sotalol, or propafenone in atrial fibrillation: which is preferred to maintain normal sinus rhythm? Pacing Clin Electrophysiol 23, 1883-1887 (2000).
11. Packer, D. L., et al. Effect of Catheter Ablation vs Antiarrhythmic Drug Therapy on Mortality, Stroke, Bleeding, and Cardiac Arrest Among Patients With Atrial Fibrillation: The CABANA Randomized Clinical Trial. JAMA 321, 1261-1274 (2019).
12. Goldberger, J. J., et al. Evaluating the Atrial Myopathy Underlying Atrial Fibrillation: Identifying the Arrhythmogenic and Thrombogenic Substrate. Circulation 132, 278-291 (2015).
13. Nattel, S. & Harada, M. Atrial remodeling and atrial fibrillation: recent advances and translational perspectives. J Am Coll Cardiol 63, 2335-2345 (2014).
14. Gaspo, R., Bosch, R. F., Bou-Abboud, E. & Nattel, S. Tachycardia-induced changes in Na+current in a chronic dog model of atrial fibrillation. Circ Res 81, 1045-1052 (1997).
15. Gemel, J., et al. Connexin40 abnormalities and atrial fibrillation in the human heart. J Mol Cell Cardiol 76, 159-168 (2014).
16. Gemel, J., et al. Degradation of a connexin40 mutant linked to atrial fibrillation is accelerated. J Mol Cell Cardiol 74, 330-339 (2014).
17. Hanif, W., et al. Left atrial remodeling, hypertrophy, and fibrosis in mouse models of heart failure. Cardiovasc Pathol 30, 27-37 (2017).
18. Park, D. S., et al. Ionic Mechanisms of Impulse Propagation Failure in the FHF2-Deficient Heart. Circ Res 127, 1536-1548 (2020).
19. Yamaguchi, N., et al. Cardiac Pressure Overload Decreases ETV1 Expression in the Left Atrium, Contributing to Atrial Electrical and Structural Remodeling. Circulation (2020).
20. Niederreither, K., et al. Embryonic retinoic acid synthesis is essential for heart morphogenesis in the mouse. Development 128, 1019-1031 (2001).
21. Ryckebusch, L., et al. Retinoic acid deficiency alters second heart field formation. Proc Natl Acad Sci USA 105, 2913-2918 (2008).
22. Yutzey, K. E., Rhee, J. T. & Bader, D. Expression of the atrial-specific myosin heavy chain AMHC1 and the establishment of anteroposterior polarity in the developing chicken heart. Development 120, 871-883 (1994).
23. Zhang, Q., et al. Direct differentiation of atrial and ventricular myocytes from human embryonic stem cells by alternating retinoid signals. Cell Res 21, 579-587 (2011).
24. Lee, J. H., Protze, S. I., Laksman, Z., Backx, P. H. & Keller, G. M. Human Pluripotent Stem Cell-Derived Atrial and Ventricular Cardiomyocytes Develop from Distinct Mesoderm Populations. Cell Stem Cell 21, 179-194 e174 (2017).
25. Cyganek, L., et al. Deep phenotyping of human induced pluripotent stem cell-derived atrial and ventricular cardiomyocytes. JCI Insight 3 (2018).
26. Yang, N., et al. Cardiac retinoic acid levels decline in heart failure. JCI Insight 6 (2021).
27. He, Y., et al. All-trans retinoic acid inhibited angiotensin II-induced increase in cell growth and collagen secretion of neonatal cardiac fibroblasts. Acta Pharmacol Sin 27, 423-429 (2006).
28. Sierra-Mondragon, E., et al. All-trans retinoic acid ameliorates inflammatory response mediated by TLR4/NF-kappaB during initiation of diabetic nephropathy. J Nutr Biochem 60, 47-60 (2018).
29. Hsu, J., et al. Genetic Control of Left Atrial Gene Expression Yields Insights into the Genetic Susceptibility for Atrial Fibrillation. Circ Genom Precis Med 11, e002107 (2018).
30. Singh, A., Addetia, K., Maffessanti, F., Mor-Avi, V. & Lang, R. M. LA Strain for Categorization of LV Diastolic Dysfunction. JACC Cardiovasc Imaging 10, 735-743 (2017).
31. Ichiki, H., et al. The role of infection in the development of non-valvular atrial fibrillation: up-regulation of Toll-like receptor 2 expression levels on monocytes. J Cardiol 53, 127-135 (2009).
32. Kajiya, T., et al. Toll-like receptor 2 expression level on monocytes in patients with viral infections: monitoring infection severity. J Infect 57, 249-259 (2008).
33. Wang, J., et al. TLR2 was overexpressed independent of IL-6 in patients with valvular atrial fibrillation. J Biomed Res 25, 178-184 (2011).
34. Yao, C., et al. Enhanced Cardiomyocyte NLRP3 Inflammasome Signaling Promotes Atrial Fibrillation. Circulation 138, 2227-2242 (2018).
35. Cheng, T., Wang, X. F., Hou, Y. T. & Zhang, L. Correlation between atrial fibrillation, serum amyloid protein A and other inflammatory cytokines. Mol Med Rep 6, 581-584 (2012).
36. Jang, H. M., et al. TLR2 and the NLRP3 inflammasome mediate IL-1beta production in Prevotella nigrescens-infected dendritic cells. Int J Med Sci 18, 432-440 (2021).
37. Martinon, F., Burns, K. & Tschopp, J. The inflammasome: a molecular platform triggering activation of inflammatory caspases and processing of proIL-beta. Molecular cell 10, 417-426 (2002).
38. Shi, Y., Li, D., Tardif, J. C. & Nattel, S. Enalapril effects on atrial remodeling and atrial fibrillation in experimental congestive heart failure. Cardiovasc Res 54, 456-461 (2002).
39. Yoon, N., et al. Beneficial effects of an angiotensin-II receptor blocker on structural atrial reverse-remodeling in a rat model of ischemic heart failure. Exp Ther Med 5, 1009-1016 (2013).
40. Paik, J., et al. Inhibition of retinoic acid biosynthesis by the bisdichloroacetyldiamine WIN 18,446 markedly suppresses spermatogenesis and alters retinoid metabolism in mice. J Biol Chem 289, 15104-15117 (2014).
41. Stoppie, P., et al. R115866 inhibits all-trans-retinoic acid metabolism and exerts retinoidal effects in rodents. J Pharmacol Exp Ther 293, 304-312 (2000).
42. Park, D. S., et al. Fhf2 gene deletion causes temperature-sensitive cardiac conduction failure. Nat Commun 7, 12966 (2016).
43. Shekhar, A., et al. ETV1 activates a rapid conduction transcriptional program in rodent and human cardiomyocytes. Sci Rep 8, 9944 (2018).
44. Shekhar, A., et al. Transcription factor ETV1 is essential for rapid conduction in the heart. J Clin Invest 126, 4444-4459 (2016).
45. Cooper, C. D. & Lampe, P. D. Casein kinase 1 regulates connexin-43 gap junction assembly. J Biol Chem 277, 44962-44968 (2002).
46. Himelman, E., et al. Prevention of connexin-43 remodeling protects against Duchenne muscular dystrophy cardiomyopathy. J Clin Invest 130, 1713-1727 (2020).
47. Sovari, A. A., et al. Mitochondria oxidative stress, connexin43 remodeling, and sudden arrhythmic death. Circ Arrhythm Electrophysiol 6, 623-631 (2013).
48. Liu, M., Liu, H. & Dudley, S. C., Jr. Reactive oxygen species originating from mitochondria regulate the cardiac sodium channel. Circ Res 107, 967-974 (2010).
49. Gutstein, D. E., et al. Conduction slowing and sudden arrhythmic death in mice with cardiac-restricted inactivation of connexin43. Circ Res 88, 333-339 (2001).
50. Park, D. S., et al. Genetically engineered SCN5A mutant pig hearts exhibit conduction defects and arrhythmias. J Clin Invest 125, 403-412 (2015).

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims. It is further to be understood that all values are approximate, and are provided for description. Patents, patent applications, publications, product descriptions, and protocols are cited throughout this application, the disclosures of which are incorporated herein by reference in their entireties for all purposes.

1. A method for treating atrial fibrillation in a subject in need thereof, the method comprising administering to the subject a therapeutically effective amount of a retinoid or a pharmaceutically acceptable salt thereof.

2. The method of claim 1, wherein the subject has been determined to be deficient for said retinoid.

3. A method for treating atrial fibrillation in a subject in need thereof, comprising:
a) determining the level of a retinoid in a serum sample obtained from the subject;
b) comparing the level of the retinoid determined in step (a) to a control level of said retinoid;
c) administering to the subject determined to be deficient for said retinoid based on the comparison in step (b) a therapeutically effective amount of said retinoid or a pharmaceutically acceptable salt thereof.

4. The method of claim 3, wherein the therapy with said retinoid or pharmaceutically acceptable salt thereof is continued until the subject is no longer determined to be deficient for said retinoid by repeating steps (a)-(b).

5. The method of any one of claims 1-4, wherein said retinoid is a retinoic acid.

6. The method of any one of claims 1-5, wherein said retinoid is all-trans retinoic acid (ATRA).

7. The method of any one of claims 1-6, wherein the subject has been diagnosed with heart failure.

8. The method of any one of claims 1-6, wherein the subject has been diagnosed with hypertension.

9. A method for preventing or reversing atrial remodeling in a subject in need thereof, the method comprising administering to the subject a therapeutically effective amount of a retinoid or a pharmaceutically acceptable salt thereof.

10. The method of claim 9, wherein the subject has been determined to be deficient for said retinoid.

11. A method for preventing or reversing atrial remodeling in a subject in need thereof, comprising:
a) determining the level of a retinoid in a serum sample obtained from the subject;
b) comparing the level of the retinoid determined in step (a) to a control level of said retinoid;
c) administering to the subject determined to be deficient for said retinoid based on the comparison in step (b) a therapeutically effective amount of said retinoid or a pharmaceutically acceptable salt thereof.

12. The method of claim 11, wherein the therapy with said retinoid or pharmaceutically acceptable salt thereof is continued until the subject is no longer determined to be deficient for said retinoid by repeating steps (a)-(b).

13. The method of any one of claims 9-12, wherein said retinoid is a retinoic acid.

14. The method of any one of claims 9-13, wherein said retinoid is all-trans retinoic acid (ATRA).

15. The method of any one of claims 9-14, wherein the subject is diagnosed with acute onset heart failure.

16. The method of any one of claims 1-15, wherein the subject is human.

17. The method of any one of claims 1-16, wherein the subject is more than 65 years of age.

18. The method of any of claims 1-17, wherein the retinoid or pharmaceutically acceptable salt thereof is administered or delivered to the atrium.

19. The method of any one of claims 1-18, wherein the retinoid or pharmaceutically acceptable salt thereof is administered in combination with one or more additional therapeutic agents.
